(19) United States
(12) Patent Application Publication
(10) Pub. No.: US 2024/0017232 A1
(43) Pub. Date: Jan. 18, 2024

(54) COUPLED FLUIDIZED BEDS REACTOR-REGENERATOR APPARATUS FOR CATALYTIC DEHYDROGENATION OF PROPANE

(71) Applicant: China University of Petroleum-Beijing, Beijing (CN)

(72) Inventors: Mengxi LIU, Beijing (CN); Chunxi LU, Beijing (CN); Chunming XU, Beijing (CN); Guiyuan JIANG, Beijing (CN); Jian LI, Beijing (CN)

(21) Appl. No.: 18/354,111

(22) Filed: Jul. 18, 2023

(30) Foreign Application Priority Data
Jul. 18, 2022 (CN) 202210841189.1

(51) Int. Cl. B01J 8/26; B01J 8/24; B01J 8/00; B01D 45/16; B01D 45/04; B01D 3/38

(52) U.S. Cl. CPC B01J 8/26; B01J 8/24; B01J 8/0015; B01J 8/005; B01D 45/16; B01D 45/04; B01D 3/38; B01J 2208/00769; B01J 2208/00893

(57) ABSTRACT

A coupled fluidized beds reactor-regenerator apparatus for catalytic dehydrogenation of propane. The fluidized bed reactor comprising a raw material delivery system, a pre-rising system, a reaction system, a gas-solid separation system and an internal circulation pipeline, the reaction system includes a conical riser and a turbulent bed reactor; the raw material delivery system, the pre-rising system, the conical riser, the turbulent bed reactor, and the gas-solid separation system are consecutively connected in this order from bottom to top; the bottom outlet of the gas-solid separation system is connected to the inlet of the internal circulation pipeline, and the outlet of the internal circulation pipeline is connected to the raw material delivery system and/or the reaction system. The coupled fluidized beds reactor-regenerator apparatus for catalytic dehydrogenation of propane includes the fluidized bed reactor, a gas-solid airlift loop regenerator, a recirculation inclined pipe and a regeneration inclined pipe.

### CROSS-REFERENCE TO RELATED APPLICATIONS

This application claims priority to Chinese Patent Application No. 202210841189.1, filed on Jul. 18, 2022, which is hereby incorporated by reference in its entirety.

### TECHNICAL FIELD

The present disclosure relates to the technical field of fluidized bed apparatus for catalytic dehydrogenation of propane, in particular to a coupled fluidized beds reactor-regenerator apparatus for catalytic dehydrogenation of propane.

### BACKGROUND OF ART

Propylene is an important organic chemical raw material of which consumption is second only to ethylene, and is the main raw material for the production of downstream products such as propylene oxide, polypropylene, acrylic acid, acrylonitrile, and butanol. With the rapid expansion and development of the chemical industry, the demand for the chemicals such as propylene oxide, polypropylene and acrylic acid in the chemical industry is increasing. Moreover, as the main upstream raw material, the demand for propylene is also increasing. At present, the production process of propylene is mainly divided into two categories: traditional process routes and novel process routes. The traditional process route is mainly based on steam cracking and catalytic cracking, both of which have the disadvantages of high energy consumption and low selectivity. The coal-to-olefins, among the novel process routes, also has high energy consumption and low selectivity. The olefin disproportionation and olefin cracking processes are seldom used because of high requirements on raw materials, while the catalytic dehydrogenation process of propane is being widely used and industrialized during these years, because of the characteristics of simple raw materials and good singleness. The so-called catalytic dehydrogenation process of propane refers to converting propane into propylene and hydrogen through catalytic heat absorption at a temperature of 500-650° C. and a pressure of 0.1-3.0 MPa.

The Catofin process (CA2968086A1/C, CN105693450A/B) and Oleflex process (US2011/0230698A1, CN102811958A) are often used in catalytic dehydrogenation of propane. The advantages of these two processes are that the back mixing is low, the catalyst is not prone to wear, and the single-pass conversion rate is high. But the two processes also have disadvantages such as low heat transfer efficiency of the bed, difficulty to control the temperature, the complicated heating means and high energy as consumed. The Catofin process also has problems such as difficult desorption of the adsorbent, and high cost for equipment and operation. In addition, the Oleflex process uses a platinum noble metal catalyst, which has a high cost and high requirements on raw materials.

The conventional fluidized bed reactor for catalytic dehydrogenation of propane has the following problems.

(1). The reaction for catalytic dehydrogenation of propane generally needs to ensure sufficient reaction time (5 to 15 s), and meanwhile it is necessary to ensure low gas back-mixing, high gas-solid contact efficiency and low catalyst wear rate, so as to ensure high product yield and high selectivity. If a single form of fluidized bed reactor is used, in order to ensure sufficient reaction time, only a bubbling or turbulent fluidized bed reactor can be used, but the bed reactor cannot meet the requirements of both long reaction time and low back-mixing; if a riser reactor is used, although the back-mixing can be reduced, the sufficient reaction time cannot be ensured due to height limitation.

(2). A fast fluidized bed reactor as the reactor for catalytic dehydrogenation of propane has problems such as uneven temperature distribution inside the reactor, serious back-mixing, low yield, and poor selectivity. However, if a dense phase bed is used as the reactor, there is more serious back-mixing in the reactor, which is equivalent to a continuous stirred tank reactor. The carbon on the catalyst is equivalent to that of the recirculation catalyst, and the activity is low, which is not conducive to improving the conversion rate and selectivity of the reaction. In addition, the conventional dense phase reactor has a poor effect of gas-solid contact, thus the bubbles coalesce and grow rapidly in the bed, the gas-solid contact area is small, and the gas is easy to mix back, which is not conducive to improving the conversion rate and selectivity of the reaction. In addition, no matter whether a fast fluidized bed or a dense phase bed is used, the gas will entrain a certain amount of catalyst into the dilute phase above the bed, and a significant secondary reaction will occur between the gas and the catalyst in the dilute phase (consuming product gas or generating unnecessary gas impurities), thereby reducing product yield and selectivity.

(3). When a riser with constricted diameter is used as the reactor, the degree of back-mixing of the gas can be reduced, but the inlet of the riser must ensure sufficient gas velocity (not less than 8 m/s) to ensure the smooth flow of the catalyst. Because of constricted neck in the outlet, in addition to the fact that the reaction for catalytic dehydrogenation of propane is a molecule increased reaction (the gas flow will increase as the reaction progresses), the gas velocity at the outlet of the riser is too high (up to 15 m/s), resulting in serious wear of the catalyst, while failing to ensure sufficient reaction time; and the riser reactor is characterized by a catalyst concentration (approximately 100 kg/m 3) is much lower than that of the bed reactor (about 500 to 800 kg/m 3), so the reaction proceeds relatively slowly.

(4). In the existing reactions for catalytic dehydrogenation of propane, the outlet of the reactor is directly connected to a cyclone separator, and all the catalyst will enter the cyclone separator with the gas. Since the tangential speed in the cyclone separator is up to 40 m/s or more, it will cause significant catalyst wear.

(5). The coke yield of the reaction for catalytic dehydrogenation of propane is low, only about 1%. The amount of coke attached to the catalyst after one pass of catalytic dehydrogenation reaction is small. When the catalyst directly enters the regenerator after passing through the reactor once, not only the regeneration temperature is low and the coke-burning regeneration effect is poor, but also the amount of catalyst is large, and the energy consumption for delivery is high. In addition, the coke content on the catalyst is too low, and the heat generated by combustion cannot meet the needs of the reaction.

(6). In sum, the fluidized bed reactors for catalytic dehydrogenation of propane proposed so far have problems such as single reactor type, serious back-mixing, easy wear of the catalyst, and poor selectivity. Severe back-mixing will lead to more secondary reactions in the reactor, thereby reducing the selectivity of propane dehydrogenation. Noble metals are often used as the catalyst, and long-term wear of the catalyst will shorten their lifespan, resulting in greatly increased catalyst usage costs. The energy consumption of the regeneration process of the catalyst is also very high.

Therefore, there is an urgent need to provide a new type of apparatus for catalytic dehydrogenation of propane, which can solve the technical defects of the existing fluidized bed reactor for catalytic dehydrogenation of propane, such as strong back-mixing and easy wear of the catalyst, and further reduce the construction and usage costs of the fluidized bed process for catalytic dehydrogenation of propane, while achieving the object of energy saving and consumption reduction.

### SUMMARY

In order to solve the above technical problems, the present disclosure provides a coupled fluidized beds reactor-regenerator apparatus for catalytic dehydrogenation of propane. The fluidized bed reactor in the coupled apparatus can effectively solve the problems existing in the existing reactor for catalytic dehydrogenation of propane, such as strong back-mixing, easy wear of catalyst, significant secondary reaction in the dilute phase, and high energy consumption.

In order to achieve the above objects, the present disclosure provides a fluidized bed reactor comprising a raw material delivery system, a pre-rising system, a reaction system, a gas-solid separation system and an internal circulation pipeline;
wherein the reaction system comprises a conical riser and a turbulent bed reactor, and the cross-sectional diameter of the conical riser gradually increases from the inlet to the outlet (i.e., a neck expansion structure from inlet to outlet);
the raw material delivery system, the pre-rising system, the conical riser, the turbulent bed reactor, and the gas-solid separation system are consecutively connected in this order from bottom to top, wherein the bottom of the gas-solid separation system is provided with an outlet connected to the inlet of the internal circulation pipeline, while the outlet of the internal circulation pipeline is connected to the raw material delivery system and/or the reaction system (the outlet of the internal circulation pipeline is preferably connected to the turbulent bed reactor), and the gas-solid separation system is provided with a product gas outlet.

According to a specific embodiment of the present disclosure, the pre-rising system, the conical riser, the turbulent bed reactor, and the gas-solid separation system are generally arranged coaxially.

According to a specific embodiment of the present disclosure, the raw material delivery system is used to deliver reaction raw materials such as propane and a catalyst to the pre-rising system. The raw material delivery system may be a conventional delivery device, such as a raw material delivery pipeline.

According to a specific embodiment of the present disclosure, the pre-rising system is used to mix the reaction raw materials and a catalyst (including the catalyst delivered together with the raw gas, the catalyst delivered by the internal circulation pipeline, and the catalyst delivered by the regeneration inclined pipe and the like) homogeneously and to form a plug flow pattern with an initial velocity upwards.

According to a specific embodiment of the present disclosure, the raw material delivery system generally extends from the bottom of the pre-rising system into the interior of the pre-rising system. The bottom of the pre-riser may be provided with a ring distributor. The ring distributor is generally located below the outlet of the raw material delivery system. In some specific embodiments, the ring distributor can deliver fluidization gas (inert gas, circulating propane gas, and the like) to the pre-rising system in the direction from bottom to top, so as to provide the reaction raw materials with the power for the upward movement.

According to a specific embodiment of the present disclosure, the outlet of the pre-rising system may have a structure of constricted neck, that is, a tapered shape with a thin top and a thick bottom, so as to connect with the inlet of the conical riser.

According to a specific embodiment of the present disclosure, the pre-rising system may specifically be a device such as a pre-riser. Specifically, the pre-riser may have a cylindrical structure.

In a specific embodiment of the present disclosure, the conical riser serves as a reactor for catalytic dehydrogenation of propane. On one hand, the conical riser has the characteristics of an plug flow, which allows the catalyst flow upward along the axis of the riser under the lifting effect of the gas, and promotes the uniform distribution of temperature in the radial direction and the uniform distribution of the catalyst concentration in the axial direction inside the reactor, so that the back-mixing of gas and catalyst can be reduced during the reaction and improve the selectivity of products. On the other hand, the catalytic dehydrogenation of propane is a molecule increased reaction, and the volume of the gas gradually increases as the reaction progresses. The conical riser has a structure of thick top and thin bottom, and can ensure that the apparent velocity of the gas and the catalyst does not change significantly. As compared with risers with an equal diameter and risers with thin top and thick bottom, the conical riser used in the present disclosure has a slower material gas velocity, which not only helps to avoid the wear of the catalyst, but also increases the concentration of the catalyst in the riser to maintain a sufficient ratio of catalyst to oil to avoid the problem of catalyst concentration reduction caused by increased gas flow and increased carrying capacity, and ensure the conversion rate of the reaction. In addition, by adopting the conical riser with an expanded neck structure, it is beneficial to the connection between the conical riser and the turbulent bed reactor with a larger diameter. The increase in the outlet area of the conical riser is conducive to the uniform distribution of the gas and solid phases output from the conical riser to the turbulent bed reactor to avoid a dead zone in the reaction bed. In the conical riser of the present disclosure, without adding additional rising medium, the catalyst can be risen by only the flow of the propane raw material, effectively reducing the load and energy consumption of the reactor.

According to a specific embodiment of the present disclosure, in the fluidized bed reactor of the present disclosure, through the interaction between the turbulent bed reactor and the conical riser, the back-mixing of the gas phase and the catalyst during the reaction can be effectively reduced while ensuring sufficient reaction time, which is beneficial to provide the product selectivity and yield.

According to a specific embodiment of the present disclosure, the angle between the wall generatrix of the conical riser and the central vertical line of the conical riser is generally controlled between 0° and 10°.

According to a specific embodiment of the present disclosure, the ratio of the outlet diameter to the inlet diameter of the conical riser is generally controlled to be less than or equal to 3 and greater than 1.

In a specific embodiment of the present disclosure, the stream output from the conical riser can stay for a long time after entering the turbulent bed reactor, so that the catalyst and the raw materials can be reacted for a longer time, which is conducive to improving the conversion rate of the reaction. Through the interaction between the turbulent bed reactor and the conical riser, the back-mixing of the gas phase and the solid phase during the reaction can be effectively reduced while ensuring sufficient reaction time between the gas phase and the solid phase, which is conducive to providing the product selectivity and yield.

According to a specific embodiment of the present disclosure, the bottom of the turbulent bed reactor is provided with a first perforated distribution plate, which can evenly distribute the gas and catalyst particles entering the turbulent bed reactor, avoiding local aggregation and plugging. In some specific embodiments, the aperture of the first perforated distribution plate is 050 mm to 0250 mm, preferably 0100 mm to 0250 mm, for example 0100 mm.

According to a specific embodiment of the present disclosure, in the direction from the conical riser to the turbulent bed reactor, the inlet section of the turbulent bed reactor has a expanded neck structure, so that the size of the connection between the turbulent bed reactor and the conical riser is similar, providing enough space for the setting of the first perforated distribution plate, and allowing the gas and catalyst evenly distributed on the first perforated distribution plate to gradually diffuse to the entire cross-section of the turbulent bed reactor, avoiding the formation of dead zones. In some specific embodiments, the opening area of the first perforated distribution plate is smaller than the cross-sectional area of the outlet of the conical riser, so the gas and catalyst will be accelerated to a certain extent when passing through the first perforated distribution plate. For example, the velocity of the gas and catalyst at the outlet of the conical riser is 8-12 m/s, and the velocity can reach 10 m/s or more when passing through the first perforated distribution plate.

According to a specific embodiment of the present disclosure, the turbulent bed reactor may be further provided with one or more layers of grids generally located above the first perforated distribution plate, and arranged in layers along the vertical direction. The grids can effectively break the air bubbles and strengthen the mass transfer between gas and solid. The multi-layer grids can also divide the turbulent bed reactor into multiple zones in series in the axial direction, thereby reducing the back-mixing phenomenon and improving the product selectivity. It can not only reduce the secondary reaction, but also improve the uniformity of temperature distribution in the reaction system, avoid local high temperature, and improve the selectivity of catalytic dehydrogenation of propane. The grids used in the present disclosure can be cross-flow grids or conventional grids.

According to a specific embodiment of the present disclosure, the distance between the lowermost layer of grid and the first perforated distribution plate is generally controlled to be greater than or equal to 500 mm. When the gas and solid phases pass through the first perforated distribution plate at a fast speed (such as 10 m/s or more), by controlling the distance between the first perforated distribution plate and the adjacent grid, the bubbles in the gas and solid phases flowing out of the first perforated distribution plate can be fully grown and broken, and the wear of the catalyst on the grids caused by too close distance therebetween can be avoided.

According to a specific embodiment of the present disclosure, the interior space of the turbulent bed reactor will generally form a catalyst bed. Specifically, during the reaction for catalytic dehydrogenation of propane, the catalyst particles in the fluidized state usually form a catalyst bed in the turbulent bed reactor upwards from the bottom (specifically, above the first perforated distribution plate). During the fluidization process, the catalyst particles above the catalyst bed are in a fluctuating state, forming a splash zone. In a specific embodiment, the height of the catalyst bed is related to the amount of catalyst in the turbulent bed reactor. In the present disclosure, the height of the catalyst bed is generally 2 m to 10 m, such as 2 m to 6 m, or 3 m to 10 m, and the splash zone is located above the catalyst bed and has a height of 1500 mm to 6000 mm.

According to a specific embodiment of the present disclosure, at least the uppermost layer of grid is located inside the catalyst bed, i.e., the uppermost layer of grid is generally completely submerged in the catalyst bed. Further preferably, the distance between the top of the uppermost layer of grid and the upper surface of the catalyst bed is controlled to be more than or equal to 500 mm.

In some specific embodiments, the openings of the grids may be rectangular, square, or the like. Correspondingly, the side length of the openings in the grids is generally 100 mm to 500 mm.

According to a specific embodiment of the present disclosure, the grids may be one or more groups of cross-flow grids including two layers of grids in each group. The vertical distance between the two layers of grids in the same group is generally 300 mm or more, and the vertical distance between two adjacent groups of grids can be controlled to 500 mm to 4000 mm (for example, in FIGS. 4, A1 and A2 form a group A of cross-flow grids, while B1 and B2 form a group B of cross-flow grids, d1 and d2≥300 mm, and d3 is 500 mm to 4000 mm). The vertical distance can ensure that the coalesced air bubbles generated between two layers of cross-flow grids are broken in time.

According to a specific embodiment of the present disclosure, the reaction system may be configured such that the reaction system is composed of a conical riser and a turbulent fluidized bed reactor, wherein the conical riser has a conical structure of cross-sectional diameter gradually increasing from the inlet to the outlet, while the inlet of the turbulent bed reactor is connected to the outlet of the conical riser and is a expanded neck structure, and the first perforated distribution plate is arranged at the inlet of the turbulent bed reactor. The turbulent bed reactor is provided with one or more groups of cross-flow grids in the space range greater than or equal to 500 mm above the first perforated distribution plate and greater than or equal to 500 mm below the surface of the catalyst bed, wherein each group of cross-flow grids has two layers, and each layer of cross-flow grid is arranged in layers along the vertical direction, and the vertical distance between the two layers of grids in the same group is greater than or equal to 300 mm so that the bubble size is reduced by two consecutive crushings. The vertical distance between two adjacent groups of grids is 500 mm to 4000 mm. The gas and solid phases enters the conical riser through the pre-rising system. The gas phase provides a rising effect for the solid phase and the gas and solid phases flow upwards in the axial direction at a steady apparent gas velocity. When the gas and solid phases enters the turbulent bed reactor from the conical riser, since the inlet of the turbulent bed reactor has an expanded neck, the flow cross-section area of the gas and solid phases is further increased. Then, the gas and solid phases are evenly distributed to the inlet section of the turbulent bed reactor by the first perforated distribution plate to avoid particle aggregation and temperature unevenness when entering the turbulent bed reactor. The gas and solid phases then move from the first perforated distribution plate to the lowermost layer of grid, and the gas phase generates bubbles when passing through the first perforated distribution plate, and the bubbles coalesce and is broken by the lowermost layer of grid. Then the gas and solid phases continues to rise, and the bubbles that coalesce during rising are broken by the grids layer by layer in a continuous manner. The present disclosure uses a reaction system coupled by a conical riser and a turbulent bed reactor, which can effectively prevent back-mixing while ensuring sufficient reaction time between gas and solid phases, thereby improving the selectivity and conversion rate of the reaction. In addition, the present disclosure uses the conical riser to cooperate with the turbulent bed reactor, which can also save equipment space while ensuring sufficient reaction time, and ensure that the catalyst concentration remains sufficient during the reaction.

In the fluidized bed reactor of the present disclosure, the gas-solid separation system is used to carry out a gas-solid separation on the reacted gas and solid phases, and to collect the solid catalyst. The gas-solid separation system generally comprises a casing (disengagement section), and a gas collection hood, a dilute phase pipe, a low-wear gas-solid separation device and cyclone separators provided inside the casing, wherein the inlet of the gas collection hood is connected to the outlet of the turbulent bed reactor, and the gas collection hood, the dilute phase pipe, the low-wear gas-solid separation device and the cyclone separator are consecutively connected in this order.

Among them, the gas collection hood and the dilute phase pipe can quickly introduce the gas phase (including unreacted propane, product gas produced in reaction and the like) discharged from the reaction system and the catalyst entrained in the gas phase into the inlet of the low-wear gas-solid separation device, and then carry out a rapid gas-solid separation to avoid problems of secondary reaction. Meanwhile, the low-wear gas-solid separation device is installed between the dilute phase pipe and the cyclone separators, which can also allow the reacted gas and solid phases to enter the low-wear gas-solid separation device for preliminary separation before entering the cyclone separators. Through the cooperation of the two-stage separation devices, it can reduce the wear degree of the solid catalyst by the high-speed cyclone separator while ensuring a high separation effect (99% or more).

In the gas-solid separation system, the gas collection hood is generally located above the catalyst bed, specifically, above the splash zone, and the vertical distance between the gas collection hood and the upper surface of the catalyst bed is generally 1500 mm to 6000 mm.

In a specific embodiment of the present disclosure, the gas collection hood is generally conical or truncated conical, and the angle between the generatrix and the central axis of the gas collection hood is 30°-70°.

In the gas-solid separation system, the dilute phase pipe is generally located above the gas collection hood.

In the gas-solid separation system, the low-wear gas-solid separation device is provided with a solid outlet downward opened and a gas outlet upward opened, and the cyclone separator is generally located above the low-wear gas-solid separation device. The gas outlet of the low-wear gas-solid separation device is connected to the inlet of the cyclone separator, while the gas outlet of the cyclone separator is connected to the product gas outlet provided in the casing of the gas-solid separation system, and the solid outlet of the cyclone separator is downward and is connected to the inner space of the casing of the gas-solid separation system. That is, the product gas separated by the cyclone separator is discharged from the fluidized bed reactor through the product gas outlet, and the solid catalyst separated by the cyclone separator falls naturally to the bottom of the casing of the gas-solid separation system.

According to a specific embodiment of the present disclosure, the communication between the low-wear gas-solid separation device and the cyclone separator can be realized in various ways, for example: the low-wear gas-solid separation device is not directly connected to the cyclone separator, but the gas outlet of the low-wear separation device is connected to the dilute phase pipe and the outlet of the dilute phase pipe is connected to the inlet of the cyclone separator, or both the gas outlet of the low-wear separation device and the inlet of the cyclone separator are connected to the inner space of the casing of the gas-solid separation system; or the gas outlet of the low-wear gas-solid separation device and the inlet of the cyclone separator are directly connected by a pipeline; or the gas outlet of the low-wear gas-solid separation device are connected to the inlet of the cyclone separator through a socket connection.

According to a specific embodiment of the present disclosure, the gas collection hood is used to collect the reacted gas phase and the catalyst entrained in the gas phase discharged from the turbulent bed reactor. Since the catalyst particles above the catalyst bed are in a fluctuating state during fluidization, the bottom end of the gas collection hood is generally located above the splash zone, and the distance between the bottom end of the gas collection hood and the upper surface of the catalyst bed is generally controlled to 1500 mm-6000 mm, preferably 3 m-4 m.

According to a specific embodiment of the present disclosure, the outlet of the dilute phase pipe is generally arranged on the lateral side.

According to a specific embodiment of the present disclosure, the low-wear gas-solid separation device may comprise a cantilever type gas-solid fast separator or an ultra-short fast separator.

According to a specific embodiment of the present disclosure, the cantilever type gas-solid fast separator comprises a cover and multiple cantilevers located inside the cover, wherein the inlets of the cantilever are connected to the outlet of the dilute phase pipe, and the ends of the cantilever are provided with a solid outlet.

According to a specific embodiment of the present disclosure, the bottom of the cover is open, and the opening is the solid outlet of the low-wear gas-solid separation device; the top of the cover is provided with a gas outlet, and the gas outlet can specifically be in the form of a outlet pipe or the like, and the gas outlet of the cover is used as the gas outlet of the low-wear gas-solid separation device.

According to a specific embodiment of the present disclosure, the cantilever type gas-solid fast separator includes two or more cantilevers, and the inlets of the cantilevers (as the inlet of the low-wear gas-solid separation device) are respectively connected to the outlets on the lateral side of the dilute phase pipe. The end of the cantilever is provided with a solid outlet, and the solid catalyst separated by the cantilever type gas-solid fast separator is discharged from the solid outlet of the cantilever, falls along the inner wall of the cover, and then falls along the bottom opening of the cover to the bottom of the casing of the gas-solid separation system, and then are piled up to form a dense bed (also known as dense phase catalyst bed).

According to a specific embodiment of the present disclosure, the extension direction of the cantilever is generally horizontal or downward spiral.

According to a specific embodiment of the present disclosure, the radial distance between the cantilever and the cover is generally short to obtain a fast separation speed. In some specific embodiments, the horizontal distance between the outlet of the cantilever and the inner side wall of the cover can be controlled within 500 mm.

When the low-wear gas-solid separation device is a cantilever type gas-solid fast separator and a cover, the outlet on the lateral side of the dilute phase pipe is generally two or more for connecting two or more cantilevers, and the outlet of the dilute phase pipe can be square, rectangle or the like.

According to a specific embodiment of the present disclosure, the low-wear gas-solid separation device may further comprise an ultra-short fast separator. The separation time of the ultra-short fast separator can be controlled within 0.5 s, and 98% or more of the catalyst entrained in the gas can be separated. Only about 2% of the catalyst will subsequently enter the cyclone separator for the second separation, effectively reducing the wear degree of the catalyst in the cyclone separator. The inlet of the ultra-short fast separator (as the inlet of the low-wear gas-solid separation device) is connected to the outlet of the dilute phase pipe, and the bottom of the ultra-short fast separator is provided with a solid outlet downward opened (as the solid outlet of the low-wear gas-solid separation device), and the top thereof is provided with a gas outlet opened (as the gas outlet of the low-wear gas-solid separation device). The dipleg of the ultra-short fast separator is generally located above the gas collection hood.

In some specific embodiments, when the low-wear gas-solid separation device is an ultra-short fast separator, the dilute phase pipe has one or two outlets, and each outlet corresponds to an inlet of the ultra-short fast separator.

The ultra-short fast separator of the present application may be a conventional ultra-short fast separator that has a central pipe and can be connected with a dilute phase pipe in the art, for example, the horizontal gas-solid ultra-short fast device described in the disclosure Chinese patent application No. 201010281847.3, with the title of the disclosure “Horizontal type gas-solid ultra-short rapid separation apparatus with nonuniform slits on gas exhaust pipe” and the publication number CN102397725A; or the fast gas-solid separator described in “Numerical investigation of performance of a fast gas-solid separator” (Powder Technology, 2015, 275:30-38.), the entire content of the patent application and the scientific literature mentioned above is hereby incorporated by reference.

According to a specific embodiment of the present disclosure, the connection between the gas-solid separation system and the inlet of the internal circulation pipeline is located at the bottom of the casing of the gas-solid separation system, specifically, it may be located below the solid outlet of the low-wear gas-solid separator device (for example, the bottom opening of the cover, or the bottom outlet of the ultra-short fast separator).

According to a specific embodiment of the present disclosure, the inner bottom of the gas-solid separation system may be provided with a ring pipe distributor. The ring pipe distributor is generally located at the bottom of the dense phase bed and below the connection between the gas-solid separation system and the inlet of the internal circulation pipeline, and is used to allow the catalyst in the dense phase bed at the bottom of the casing of the gas-solid separation system to be in the fluidized state and easy to enter the internal circulation pipeline.

According to a specific embodiment of the present disclosure, the pre-rising system, the conical riser, the turbulent bed reactor, the gas collection hood, the dilute phase pipe, the low-wear gas-solid separation device, and the cyclone separator are generally arranged coaxially.

According to a specific embodiment of the present disclosure, in the fluidized bed reactor, the internal circulation pipeline is used to deliver the catalyst collected in the gas-solid separation system to the raw material delivery system or the reaction system. The internal circulation pipeline may have various specific forms, including:

Form 1: the internal circulation pipeline may be a first catalyst circulation pipeline, as shown in FIG. 8, where the inlet of the first catalyst circulation pipeline is connected to the gas-solid separation system, and the outlet of the first catalyst circulation pipeline is connected to the inlet of the raw material delivery system. In some specific embodiments, the first catalyst circulation pipeline is generally provided with a wear-resistant valve for controlling the connection between the first catalyst circulation pipeline and the raw material delivery system and controlling the flow of catalyst.

Form 2: the internal circulation pipeline may further comprise a first catalyst circulation pipeline having an inlet connected with the gas-solid separation system, and an outlet connected to the turbulent bed reactor, and a second catalyst circulation pipeline having an inlet connected to the turbulent bed reactor, and an outlet connected to the inlet of the raw material delivery system, as shown in FIGS. 1 and 7. In this form, the second catalyst circulation pipeline can directly deliver the catalyst with a low coke yield in the reaction system back to the raw material delivery system for catalysis and heat supply, which is beneficial to increase the catalyst concentration in the reaction system (especially beneficial to increase the catalyst concentration in the conical riser), and can reduce the load for gas-solid separation in the gas-solid separation system, thereby reducing the overall separation loss of the fluidized bed reactor. According to a specific embodiment of the present disclosure, when the turbulent bed reactor is provided with a first perforated distribution plate and grids, and the internal circulation line comprises a first catalyst circulation line and a second catalyst circulation line, the connection between the outlet of the first catalyst circulation line and the turbulent bed reactor may be specifically above the uppermost layer of grid; and the connection between the inlet of the second catalyst circulation line and the turbulent bed reactor may be specifically between the lowermost layer of grid and the first perforated distribution plate.

According to a specific embodiment of the present disclosure, the fluidized bed reactor may further include a product gas separation system.

The product gas separation system comprises a compression condensing unit, a first separation unit and a second separation unit, wherein the compression condensing unit is provided with an inlet, a gas outlet and a liquid outlet, the inlet of the compression condensing unit is connected to the product gas outlet of the fluidized bed reactor, and the gas phase outlet of the compression condensing unit is connected to the inlet of the first separation unit, and the liquid phase outlet of the compression condensing unit is connected to the inlet of the second separation unit. The first separation unit is provided with a hydrogen outlet and a light hydrocarbon outlet, and the second separation unit is provided with a propylene outlet, a propane outlet and a fuel hydrocarbon outlet. The light hydrocarbon outlet of the first separation unit is connected to the inlet of the second separation unit.

In the above product gas separation system, the compression condensing unit is used to further compress and condense the product gas discharged from the gas-solid separation system in a stage-by-stage manner. The compression condensing unit may specifically comprise multi-stage compressors, and inter-stage cooling equipment and inter-stage separation tanks arranged between the multi-stage compressors. The multi-stage compressors are used to compress the gas sequentially, the inter-stage cooling equipment is used to lower down the gas temperature and condense, and the inter-stage separation tank is used to separate the condensed liquid phase.

In the above product gas separation system, the first separation unit is used to separate hydrogen and light hydrocarbons in the gas phase, and the second separation unit is used to separate propylene, propane, and fuel hydrocarbons (including light hydrocarbons and C4+ heavier hydrocarbons).

In the above product gas separation system, the second separation unit may include two or more depropanizers to separate light hydrocarbons, propane, propylene and C4+ heavier hydrocarbons through a rectification process.

In the above product gas separation system, the propane outlet of the second separation unit is generally connected to at least one of the gas-solid separation system, the pre-rising system, and the raw material delivery system in the fluidized bed reactor. Specifically, the propane outlet of the second separation unit may be connected to at least one of the ring pipe distributor at the bottom of the gas-solid separation system, the ring distributor at the bottom of the pre-rising system, and the raw material delivery system.

The present disclosure also provides a gas-solid airlift loop regenerator, wherein the gas-solid airlift loop regenerator comprises a first regeneration system, a second regeneration system and a first stripper communicating in this order;
the first regeneration system includes a first casing and a main air distributor, a first ring pipe distributor and a first draft tube arranged inside the first casing;
wherein the main air distributor is arranged at the bottom of the first casing, the first draft tube is arranged above the main air distributor, and the first ring pipe distributor is arranged between the first draft tube and the first casing in the horizontal direction; the top of the first casing is provided with a fuel feed nozzle extending from the outside to the inside of the first casing and located above the first draft tube;
the second regeneration system includes a second casing, and a second ring pipe distributor and a combined cyclone separator located inside the second casing, wherein the combined cyclone separator is provided on the top of the second casing, and the second ring pipe distributor is provided at the bottom of the second casing, and the top of the second casing is provided with a gas outlet;
the first stripper is used to remove an oxygen-containing flue gas, and provided with a gas outlet, an inlet and a solid outlet, and the inlet of the first stripper is connected to the bottom of the second casing.

The gas-solid airlift loop regenerator provided by the present disclosure can be used for catalyst regeneration in processes such as catalytic hydrogenation of propane. Due to low coke yield in the catalytic hydrogenation reaction of propane, the coke attached to the catalyst that needs to be regenerated (hereinafter referred to as the spent catalyst or recirculation catalyst) is not enough to maintain the combustion temperature in the regenerator after burning, and the heat of the catalyst itself is not enough to meet the energy requirements of the regeneration reaction, so the present disclosure provides the fuel feed nozzle in the first regeneration system to supplement the heat of the spent catalyst, which can restore the activity of the spent catalyst on the one hand, and can bring the heat generated by burning to the fluidized bed reactor when the spent catalyst is converted into a regenerated catalyst (hereinafter referred to as regenerant), to provide the heat required for the catalytic hydrogenation reaction of propane on the other hand.

Further, the main air distributor, the first ring pipe distributor and the first draft tube provided in the first regeneration system and the second ring pipe distributor provided in the second regeneration system of the present disclosure can allow the spent catalyst to move in the form of airlift loop in the first regeneration system and the second regeneration system, and the use of gas-solid internal circulation can improve the orderliness and mixing effect of the catalyst flow, thereby effectively improving the solid content and velocity distribution, and improving the regeneration effect. Specifically, the radial mixing effect of catalyst particles in the existing industrial fluidized bed is very poor, and the radial mixing effect of particles is 10% or less of the axial effect, thus resulting in extremely high local temperature at the outlet of the fuel feed nozzle, thus forming hot spots and damaging the catalyst. However, the present disclosure can significantly strengthen the macroscopic flow of the catalyst in the radial direction by introducing a gas-solid internal circulation, in addition to the heat provided by the fuel injected above the draft tube, to enable rapid mixing of catalysts at different temperatures and achieve a uniform distribution of temperature, thereby effectively avoiding the problems of local high temperature and hot spots caused by the injection of fuel from the fuel feed nozzle. In addition, the radial flow direction of catalyst particles below the draft tube is perpendicular to the air direction of the main air distributor, which can allow the catalyst to more sufficiently contact with the air and improve the uniformity of temperature distribution.

According to a specific embodiment of the present disclosure, in the first regeneration system, the first draft tube divides the inner space of the first casing into two parts: an inner space of the first draft tube surrounded by the draft tube, and an annulus space surrounded by the first draft tube and the first casing. The main air distributor is located in the vertical projection area of the inner space of the first draft tube, and the first ring pipe distributor is located in the vertical projection area of the annular space. Further, the first ring pipe distributors may be distributed symmetrically with the central axis of the first draft tube as the axis.

According to a specific embodiment of the present disclosure, the first ring pipe distributor is generally located above the main air distributor.

According to a specific embodiment of the present disclosure, the height of the first draft tube is generally 1 m-5 m.

It is found in the present disclosure that about 80% of the gas in the ring pipe distributor may immigrate into the inside of the draft tube. In order to ensure the effect of fluidizing the catalyst in the annular space produced by the first ring pipe distributor and reduce the amount of immigrated gas, the vertical distance between the bottom end of the first draft tube and the top end of the first ring pipe distributor is generally controlled to be less than or equal to 500 mm, for example, it can be controlled to be less than or equal to 300 mm.

According to a specific embodiment of the present disclosure, the vertical distance between the first draft tube and the first main air distributor is generally greater than or equal to 300 mm.

According to a specific embodiment of the present disclosure, the vertical distance between the fuel feed nozzle and the first draft tube is generally controlled to 200 mm-1500 mm.

According to a specific embodiment of the present disclosure, generally there are two or more fuel feed nozzles. The fuel feed nozzles may be evenly distributed along the circumference of the first casing.

According to a specific embodiment of the present disclosure, a second perforated distribution plate is further provided inside the second casing, and the second perforated distribution plate is located at the bottom of the second casing, and is specifically located below the second ring pipe distributor and above the inlet of the second casing. The second perforated distribution plate can evenly distribute the gas and the spent catalyst particles entering the regeneration system, avoiding local aggregation and plugging.

According to a specific embodiment of the present disclosure, the inlet of the second casing is tapered with a thick top and a thin bottom. This expanded neck structure can provide enough space for the installation of the second perforated distribution plate, and enable the gas and catalyst passing through the second perforated distribution plate to gradually and uniformly diffuse to the entire cross-section of the second regeneration system, avoiding the formation of dead zones.

According to a specific embodiment of the present disclosure, the second regeneration system may further include a second draft tube arranged at the inner bottom of the second casing, and the second ring pipe distributor is located between the second draft tube and the second casing in the horizontal direction.

According to a specific embodiment of the present disclosure, in the second regeneration system, the second draft tube divides the inner space of the second casing into two parts: the inner space surrounded by the second draft tube, and the annulus space surrounded by the second draft tube and the second casing. The second ring pipe distributor is located in the vertical projection area of the annular space. Further, the second ring pipe distributors may be distributed symmetrically with the central axis of the second draft tube as the axis.

According to a specific embodiment of the present disclosure, the height of the second draft tube is generally 1 m-3 m.

According to a specific embodiment of the present disclosure, the vertical distance between the bottom end of the second draft tube and the second ring pipe distributor is generally controlled to be less than or equal to 500 mm, for example, less than or equal to 300 mm, so as to ensure the effect of fluidizing the catalyst in the annulus space produced by the second ring pipe distributor and reduce the amount of gas immigrated from the second ring pipe distributor to the second draft tube.

According to a specific embodiment of the present disclosure, the main air distributor, the first draft tube, the first ring pipe distributor, the second perforated distribution plate, the second ring pipe distributor, the second draft tube, the first casing and the second casing are generally arranged coaxially.

According to a specific embodiment of the present disclosure, the first ring pipe distributor and the second ring pipe distributor may be respectively connected with two gas inlet pipes to ensure sufficient gas introduction. In some specific embodiments, the fluidization gas used in the first ring pipe distributor and the second ring pipe distributor may be air or the like, which can be used to support the combustion of coke.

According to a specific embodiment of the present disclosure, the gas-solid airlift loop regenerator may further include a regenerant internal circulation pipeline, which is used to connect the first regeneration system and the second regeneration system and balance the height of the catalyst bed in the first regeneration system and the second regeneration system.

In a specific embodiment of the present disclosure, the first stripper is used to separate the gas such as oxygen that may consume propylene and propane in the mixture discharged from the second regeneration system, so as to increase the product yield and reduce the risk of explosion in the reaction system.

According to the specific embodiment of the present disclosure, the present disclosure has no special requirements on the structure of the first stripper. As long as the stripper can remove the oxygen-containing flue gas in the catalyst, it can be used as the first stripper. In some specific embodiments, the first stripper may include: a third casing and a partition member, a third draft tube, a ring pipe steam distributor and a stripping steam distributor provided inside the third casing, wherein the partition member, the third draft tube, and the stripping steam distributor are arranged in this order from top to bottom (preferably, coaxially), and correspondingly, the gas outlet, inlet and solid outlet of the first stripper are arranged in this order from top to bottom, and the ring pipe steam distributor is arranged between the third casing and the third draft tube in the horizontal direction.

According to a specific embodiment of the present disclosure, the partition member may include a baffle, a grid or the like. The arrangement form of the partition member includes one or two or more of ring and disc baffles and grids.

According to a specific embodiment of the present disclosure, the partition member includes baffle sets arranged coaxially from top to bottom, and each baffle set includes ring baffles and disc baffles arranged in this order from top to bottom.

According to a specific embodiment of the present disclosure, the solid outlet of the first stripper is used to output the regenerant particles, and the gas outlet of the first stripper is used to output the oxygen-containing flue gas. In some specific embodiments, the flue gas output from the gas outlet will entrain a small amount of the regenerant particles. At this time, the gas outlet of the first stripper can be connected to the casing of the second regeneration system, and the combined cyclone separator can be used to separate the regenerant particles entrained in the flue gas discharged from the first stripper.

According to a specific embodiment of the present disclosure, the gas-solid airlift loop regenerator may further include a catalyst activator for loading a metal active component onto the regenerant. The catalyst activator is provided with a raw material inlet, a solid outlet, a regenerant inlet and a gas outlet in this order from bottom to top. Among them, the raw material inlet is used to receive the active component raw materials (specifically, propane and metal oxide); the regenerant inlet of the catalyst activator is generally connected to the solid outlet of the first stripper for receiving the regenerant output from the first stripper; the solid outlet of the catalyst activator is used to output the regenerant loaded with the metal active component, and the gas outlet of the catalyst activator is used to discharge the gas in the catalyst activator to keep the air pressure balance.

In the industrial reaction of catalytic dehydrogenation of propane, the active component in the used catalyst is generally a metal oxide. Due to the presence of a large amount of propane in the reaction system, which belongs to the reducing atmosphere, a part of the metal oxide on the catalyst is reduced by propane and converted into metal elementary substance. The metal elementary substance turns into metal vapor at the reaction temperature and flows out of the reactor with the product gas, resulting in the loss of active components of the catalyst and decrease in activity. However, the catalyst activator provided in the present disclosure can supplement the active component lost in the catalyst during the reaction by loading the metal active component on the regenerant. In some specific embodiments, propane and metal oxide can be introduced to the catalyst activator, and the metal oxide is reduced by propane to form metal vapor, and the metal interacts with hydroxide ion or the like on the surface of the catalyst and then attaches to the catalyst to form the metal active component. The gasification and deposition of the metal in the catalyst is a dynamic equilibrium. Since the concentration of metal vapor input into the catalyst activator is much greater than the concentration of metal vapor dissipated in the propane dehydrogenation reaction, the amount of the metal deposited on the catalyst is much greater than the amount of the metal lost in the catalyst, so as to ensure that the active component of the catalyst increases after the catalyst passes through the catalyst activator, so that the catalyst activity is improved.

In some specific embodiments, a third perforated distribution plate may be provided above the raw material inlet of the catalyst activator to ensure uniform distribution of the metal oxide and propane entering the catalyst activator and avoid local accumulation and plugging.

The present disclosure further provides a coupled fluidized beds reactor-regenerator apparatus for catalytic dehydrogenation of propane including the fluidized bed reactor, the gas-solid airlift loop regenerator, a recirculation inclined pipe and a regeneration inclined pipe;

wherein the inlet of the recirculation inclined pipe is connected to the turbulent bed reactor in the fluidized bed reactor; the outlet of the recirculation inclined pipe is connected to the inlet of the first regeneration system in the gas-solid airlift loop regenerator; the inlet of the regeneration inclined pipe is connected to the solid outlet of the first stripper, and the outlet of the regeneration inclined pipe is connected to the pre-rising system in the fluidized bed reactor.

In the above coupled apparatus, the catalyst with a high coke yield discharged from the turbulent bed reactor of the fluidized bed reactor enters the gas-solid airlift loop regenerator through the recirculation inclined pipe, and sequentially passes through the first regeneration system and the second regeneration system for coke-burning regeneration. The regenerated catalyst enters the pre-rising system in the fluidized bed reactor through the regeneration inclined pipe after the first stripper removes the oxygen-containing flue gas, and participates in the propane dehydrogenation reaction as a recycled catalyst.

In the above coupled apparatus, the outlet of the raw material delivery system is generally located below the connection between the regeneration inclined pipe and the pre-rising system, or the outlet of the raw material delivery system can also be flush with the connection between the regeneration inclined pipe and the pre-rising system.

In the above coupled apparatus, the fluidized bed reactor and the gas-solid airlift loop regenerator are generally arranged in parallel in the horizontal direction to save space.

In the above-mentioned coupled apparatus, the connection between the recirculation inclined pipe and the turbulent bed reactor is generally located above the uppermost grid in the turbulent bed reactor. Further, when the outlet of the first catalyst circulation line in the fluidized bed reactor is connected to the turbulent bed reactor, as shown in FIG. 1, the inlet of the recirculation inclined pipe is generally located above the outlet of the first catalyst circulation pipeline.

In the above coupled apparatus, a second stripper may be further connected between the outlet of the recirculation inclined pipe and the inlet of the first regeneration system. The second stripper can be used to replace the product gas adsorbed in the spent catalyst, thereby increasing the product gas yield. The present disclosure has no special requirements on the structure of the second stripper, and any stripper capable of replacing propylene gas in the art can be used as the second stripper of the present disclosure.

The amount of the regenerant treated via the gas-solid airlift loop regenerator in the above coupled apparatus and entering the pre-riser for catalyst supplement cannot achieve the density (200 kg/m³ or more) required by the catalytic dehydrogenation reaction of propane. And in some embodiments of the present disclosure, by providing the second catalyst circulation line (as shown in FIG. 1), the catalyst in the turbulent bed reactor can be introduced to the pre-riser. Because the temperature of the catalyst at the bottom of the turbulent bed reactor is relatively low and the reaction temperature in the conical riser will not be significantly increased, the interaction of the catalyst supplement in the second catalyst circulation pipeline and the regenerant delivery of the gas-solid airlift loop regenerator with each other can effectively solve the problem of low catalyst content in the riser, and further increase the density of the catalyst in the riser to 200 kg/m³ or more.

In the above coupled apparatus, when the fluidized bed reactor includes a product gas separation system, the fuel hydrocarbon outlet of the second separation unit in the product gas separation system can be connected to the fuel feed nozzle of the first regeneration system in the gas-solid airlift loop regenerator to provide fuel for the regeneration process of the catalyst.

In the above coupled system, when the gas-solid airlift loop regenerator includes a catalyst activator, the solid outlet of the catalyst activator is connected to the inlet of the regeneration inclined pipe, and at this time, the first stripper, the catalyst activator, the regeneration inclined pipe and the pre-rising system are connected in this order. The regenerant delivered by the second regeneration system passes through the first stripper to remove the oxygen-containing flue gas, and then is loaded with the metal active component in the catalyst activator and then delivered to the pre-rising system through the regeneration inclined pipe to participate in the catalytic dehydrogenation reaction of propane.

In the above coupled system, the gas outlet of the catalyst activator can be connected to the gas-solid separation system in the fluidized bed reactor, and the gas discharged from the catalyst activator contains metal vapor, propane gas and entrained catalyst particles. The gas discharged from the catalyst activator is delivered to the gas-solid separation system, and the gas-solid separation can be carried out through the cyclone separator in the gas-solid separation system. The separated catalyst particles fall to the dense phase bed at the bottom, and the metal vapor, propane gas, and the like are discharged through the product gas outlet at the top of the gas-solid separation system. In some specific embodiments, the gas outlet of the catalyst activator can be connected to the ring pipe distributor in the gas-solid separation system.

In the above coupled system, the raw material inlet of the catalyst activator can be connected to the propane outlet of the second separation unit of the product gas separation system, so as to receive the propane gas separated from the second separation unit as the raw material of the metal active component.

According to a specific embodiment of the present disclosure, in the above coupled apparatus, the pre-rising system receives the propane and catalyst delivered by the raw material delivery pipeline, the catalyst delivered by the internal circulation pipeline from the gas-solid separation system and/or reaction system, and the regenerant delivered by the gas-solid airlift loop regenerator. The temperatures of the catalysts delivered by the above devices are varied, and the present disclosure can mix the catalysts of different temperatures evenly through the pre-rising system.

According to a specific embodiment of the present disclosure, when the gas-solid airlift loop regenerator includes a catalyst activator, the product gas separation system may further include a primary condensation unit and a gas-solid separation unit, wherein the product gas outlet of the fluidized bed reactor, the primary condensation unit, the gas-solid separation unit and the compression condensing unit are connected in this order.

The present disclosure also provides a process for catalytic dehydrogenation of propane carried out in the coupled apparatus, the process comprising:
delivering propane and a catalyst to the pre-rising system through the raw material delivery system, and then delivering propane and the catalyst into the conical riser and the turbulent bed reactor sequentially through the pre-rising system to carry out the catalytic dehydrogenation reaction of propane;
delivering a part of the catalyst in the turbulent bed reactor into the recirculation inclined pipe, and delivering the remaining catalyst, entrained by the gas, from the turbulent bed reactor to the gas-solid separation system for gas-solid separation, then discharging the separated gas from the gas outlet of the gas-solid separation system, allowing the separated catalyst to fall to the bottom of the casing of the gas-solid separation system to form a dense phase bed, and delivering the catalyst in the dense phase bed by the internal circulation pipeline to the raw material delivery system and/or the reaction system for recycling;
wherein, the catalyst in the recirculation inclined pipe sequentially enters the first regeneration system and the second regeneration system in the gas-solid airlift loop regenerator for coke-burning regeneration, and then is delivered to the stripper by the second regeneration system to separate an oxygen-containing flue gas to obtain the regenerant, which sequentially enters the pre-rising system through the regeneration inclined pipe, and then enters the conical riser and the turbulent bed reactor in this order to participate in the catalytic dehydrogenation reaction of propane to complete the recycling of the catalyst.

According to a specific embodiment of the present disclosure, the above process can also be carried out in a coupled apparatus formed by replacing the conical riser with a conventional riser.

According to a specific embodiment of the present disclosure, the catalyst in the raw material delivery pipeline comes from the fresh catalyst delivered from the outside on the one hand, and the catalyst delivered from the internal circulation pipeline on the other hand.

According to a specific embodiment of the present disclosure, the catalyst in the pre-rising system comes from the catalyst delivered by the raw material delivery system on the one hand, and the regenerated catalyst delivered by the gas-solid airlift loop regenerator on the other hand.

In a specific embodiment of the present disclosure, when the internal circulation pipeline of the fluidized bed reactor in the coupled apparatus only includes the first catalyst circulation pipeline, and the inlet and outlet of the first catalyst circulation pipeline are respectively connected to the gas-solid separation system and the raw material delivery system (as shown in FIG. 8), in the above process, the catalyst in the dense phase bed of the gas-solid separation system is delivered to the raw material delivery system through the first catalyst circulation pipeline.

In a specific embodiment of the present disclosure, when the internal circulation pipeline of the fluidized bed reactor in the coupled apparatus includes a first catalyst circulation pipeline and a second catalyst circulation pipeline (as shown in FIGS. 1 and 7), in the above process, the catalyst in the dense phase bed of the gas-solid separation system can be delivered to the turbulent bed reactor through the first catalyst circulation pipeline. At this time, the above process can further include: after the gas and solid phases output from the conical riser enters the turbulent bed reactor for the catalytic dehydrogenation reaction of propane, the catalyst in the turbulent bed reactor can be divided into three parts, wherein the first part of the catalyst is delivered to the raw material delivery system through the internal circulation pipeline (the first catalyst circulation pipeline), the second part of the catalyst enters the recirculation inclined pipe, and the third part of the catalyst is entrained by the gas and enters the gas-solid separation system from the turbulent bed reactor.

In some specific embodiments, the gas velocity at the outlet of the conical riser can be controlled to 8-12 m/s, for example 8-10 m/s.

In some specific embodiments, the gas velocity of the gas and solid phases discharged from the conical riser when passing through the first perforated distributor may be up to 10 m/s or more.

In some specific embodiments, in the catalytic dehydrogenation reaction of propane, the turbulent bed reactor is generally controlled to a reaction temperature of 500° C.-620° C., a reaction pressure generally of less than or equal to 1 MPa, for a reaction time generally of 5s-15s. The conical riser has a reaction temperature generally of 600-680° C., slightly higher than that in the turbulent bed reactor, a reaction pressure generally of less than or equal to 1 MPa, and a reaction time generally of 5s-15s.

In some specific embodiments, the temperature of the regeneration reaction in the first regeneration system and the second regeneration system is generally controlled to 620-750° C., such as 620° C.-700° C., 650-750° C., or the like, and the pressure of the regeneration reaction is generally less than or equal to 1 MPa.

According to a specific embodiment of the present disclosure, the gas discharged from the product gas outlet of the fluidized bed reactor in the coupled apparatus may be propylene gas containing impurities such as propane and hydrocarbons. When the fluidized bed reactor comprises a product gas separation system, the process may further comprise:
delivering the product gas discharged from the gas-solid separation system of the fluidized bed reactor to a compression condensing unit for compression condensation and gas-liquid separation, delivering the obtained gas phase into the first separation unit, and delivering the obtained liquid phase into the second separation unit;
separating the gas phase into hydrogen and light hydrocarbons in the first separation unit, discharging the hydrogen from the product gas separation system, and delivering the light hydrocarbons into the second separation unit, wherein the light hydrocarbons include C1, C2 and impurities such as liquid phase droplets;
separating the light hydrocarbons output from the first separation unit and the liquid phase output from the compression condensing unit into propylene, propane and fuel hydrocarbons including C1, C2 and C4+ in the second separation unit, and discharging propylene from the product gas separation system, and collecting it as the product gas;
delivering the propane discharged from the second separation unit into at least one of the gas-solid separation system, the pre-rising system, and the raw material delivery system.

According to a specific embodiment of the present disclosure, when the fuel hydrocarbon outlet of the second separation unit in the product gas separation system is connected to the fuel feed nozzle of the first regeneration system, the above process may further include the following process: delivering the fuel hydrocarbons discharged from the second separation unit into the fuel feed nozzle of the first regeneration system as fuel; preferably, the fuel hydrocarbons are subjected to a decompression treatment before entering the first regeneration system.

In the above separation process, the mixed product gas coming out of the top of the fluidized bed reactor firstly undergoes stagewise compression, condensation, and gas-liquid separation in the compression condensing unit, and the gas phase containing hydrogen and light hydrocarbons separated from the compression condensing unit enters the first separation unit, and the liquid phase containing propane, propylene and other heavier hydrocarbons C4+ separated from the compression condensing unit enters the second separation unit; wherein the first separation unit can separate the hydrogen and light hydrocarbons by hydrogen separation processes such as pressure swing adsorption, and then the hydrogen flows out of the device as a product gas, and the light hydrocarbons (containing impurities such as liquid phase droplets) enter the second separation unit. The second separation unit can separate light hydrocarbons, propane, propylene and C4+ heavier hydrocarbons respectively from the liquid phase and light hydrocarbons with a rectification process. Among them, light hydrocarbons (C1, C2) and C4+ heavier hydrocarbons are decompressed and then introduced into the fuel feed nozzle of the loop regenerator as fuel, and propylene is collected as a product gas. There are many ways for recycling propane. For example, it can enter the ring pipe distributor at the bottom of the gas-solid separation system as a fluidizing gas to promote the outflow of the catalyst in the dense phase bed, or enter the ring pipe distributor of the pre-rising system as a fluidizing gas to promote the movement of raw materials (propane, catalyst) to the conical riser, or be mixed with fresh propane and enter the raw material delivery system to participate in the reaction as a raw gas. The propane can also be recycled in two or three ways as above at the same time.

According to a specific embodiment of the present disclosure, when the gas-solid airlift loop regenerator includes a catalyst activator, the process may include: allowing the regenerant to enter the catalyst activator through the regeneration inclined pipe, where a metal active component is loaded thereon, and then allowing the regenerant to enter the pre-rising system through the regeneration inclined pipe.

Further, in the case where the gas-solid airlift loop regenerator includes a catalyst activator, when the product gas separation system includes a primary condensation unit and a gas-solid separation unit, the above process may further include: firstly condensing the metal vapor in the product gas discharged from the gas-solid separation system of the turbulent bed reactor by the primary condensation unit to below the vaporization point and converting it to metal particles, then separating the metal particles from the product gas in the gas-solid separation unit, oxidizing and delivering the separated metal particles to the catalyst activator, and delivering the separated gas into the next stage of compression condensing unit for compression condensation. The separation of metal particles can prevent the product gas from being polluted on the one hand, and on the other hand, they can be sent to the catalyst activator for recycling.

The beneficial effects of the present disclosure are as follows.

1. The fluidized bed reactor provided by the present disclosure can ensure that the velocity of gas and catalyst does not change substantially in the axial direction by introducing a conical riser, which ensures the plug flow of the gas and solid phases and uniform distribution of the catalyst concentration in the axial direction, and is beneficial to improve the product yield and selectivity.

2. The fluidized bed reactor provided by the present disclosure uses a conical riser and a turbulent bed reactor as a coupled reaction system, which can not only ensure sufficient reaction time for the raw materials, but also reduce the back-mixing phenomenon of the gas and solid phases and the catalyst wear degree. Meanwhile, it can also stabilize the flow rate of the catalyst, increase the catalyst concentration in the reaction system, and improve the product selectivity and yield.

3. The fluidized bed reactor provided by the present disclosure utilizes the characteristic of low coke yield in the catalyst during the catalytic dehydrogenation of propane, and recycles directly or indirectly the catalyst with low coke yield to the reaction system by providing an internal circulation pipeline, which can improve the utilization rate of the catalyst, and increase the catalyst concentration in the reaction system, reduce energy consumption for catalyst regeneration and avoid catalyst loss.

4. The gas-solid separation system in the fluidized bed reactor provided by the present disclosure may achieve the rapid separation of the catalyst and the reaction product, avoiding problems of secondary reaction and coking, while avoiding catalyst wear.

5. The gas-solid airlift loop regenerator provided by the present disclosure can not only strengthen the regeneration effect, but also strengthen the radial flow of the spent catalyst to promote the temperature exchange between the spent catalyst and achieve uniform temperature distribution in the regeneration system, by supplementing heat to the catalyst with low coke yield using a fuel and controlling the gas-solid circumfluence movement of the spent catalyst.

6. In the present disclosure, by combining the fluidized bed reactor with the gas-solid airlift loop regenerator, the coupled apparatus thus obtained can effectively prevent the gas-solid back-mixing, reduce the catalyst wear, improve the catalyst utilization rate, and increase the reaction selectivity and yield. Meanwhile, it can reduce the construction and usage costs of the fluidized bed process for catalytic dehydrogenation of propane, achieve the purpose of saving energy and reducing consumption, and has a high prospect of industrial promotion.

### BRIEF DESCRIPTION OF DRAWINGS

FIG. 1 is a schematic structural view of the coupled apparatus in Example 1.

FIG. 2 is a schematic structural view of the fluidized bed reactor in Example 1.

FIG. 3 is a schematic structural view of a part of the fluidized bed reactor in Example 1.

FIG. 4 is a schematic structural diagram of the cross-flow grids of the present disclosure.

FIG. 5 is a schematic structural view of the gas-solid airlift loop regenerator in Example 1.

FIG. 6 is a schematic structural view of the first stripper in Example 1.

FIG. 7 is a schematic structural view of the coupled apparatus in Example 2.

FIG. 8 is a schematic structural view of the coupled apparatus in Example 3.

FIG. 9 is a schematic structural view of the coupled apparatus in Example 5.

FIG. 10 is a schematic structural view of the coupled apparatus in Example 7.

FIG. 11 is a schematic structural view of the catalyst activator in Example 7.

### DESCRIPTION OF NUMERALS

Raw material delivery pipeline 1, ring distributor 2, regeneration inclined pipe 3, pre-riser 4, conical riser 5, first perforated distribution plate 6, first catalyst circulation pipeline 7, recirculation inclined pipe 8, turbulent bed reactor 9, cross-flow grids 10, gas collection hood 11, ring pipe distributor 12, dilute phase pipe 13, cantilever type separator 14, cover secondary cyclone separator 16, product gas outlet 17, second catalyst circulation pipeline 34, outlet pipe 35 at the top of the cover, ultra-short horizontal fast separator 36, central pipe 361 of the ultra-short horizontal fast separator, gas phase outlet pipe 362 of the ultra-short horizontal fast separator, catalyst bed 90, upper surface 901 of the catalyst bed, and splash zone 91.
Main air distributor 18, first ring pipe distributor 19, first draft tube 20, regenerant circulation pipeline 21, fuel feed nozzle 22, second perforated distribution plate 23, second ring pipe distributor 37, second draft tube 38, combined cyclone separator 24, gas outlet 25 of the second regeneration system, inlet 26 of the first stripper, ring baffle 27, disc baffle 28, third draft tube 29, ring pipe steam distributor 30, stripping steam distributor 31, solid outlet 32 of the first stripper, oil and gas outlet pipeline 33.
Primary condensation unit 44, gas-solid separation unit 45, compression condensing unit 41, first separation unit 42, and second separation unit 43.
Catalyst activator 50, third perforated distribution plate 51, raw material inlet 52, regenerant inlet 53, solid outlet 54, and gas outlet 55.

### DETAILED DESCRIPTION OF EMBODIMENTS

In order to have a clearer understanding of the technical features, purposes and beneficial effects of the present disclosure, the technical solutions of the present disclosure will now be described below in details, but it should not be construed as limiting the implementable scope of the present disclosure.

### Example 1

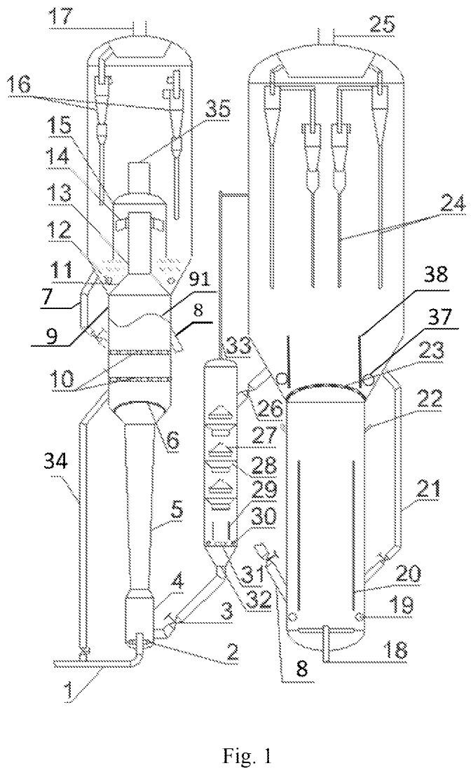

This example provides a coupled fluidized beds reactor-regenerator apparatus for catalytic dehydrogenation of propane. As shown in FIG. 1, the coupled apparatus includes a fluidized bed reactor, a gas-solid airlift loop regenerator, a recirculation inclined pipe 8 and a regeneration inclined pipe 3.

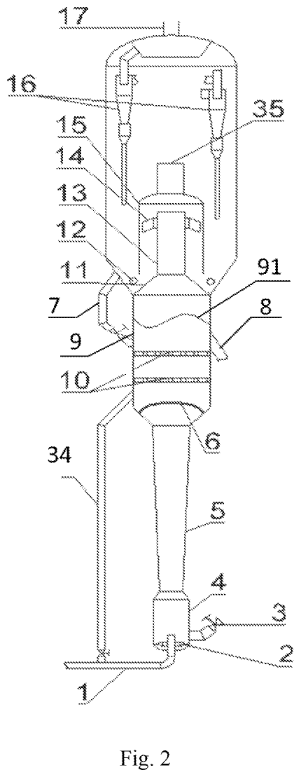

As shown in FIG. 2, the fluidized bed reactor comprises a raw material delivery system, a pre-rising system, a reaction system, a gas-solid separation system and an internal circulation pipeline.

The raw material delivery system is used to deliver reaction raw materials to the pre-rising system. In this example, the raw material delivery system is the raw material delivery pipeline 1. The raw material delivery pipeline 1 is used to receive reaction raw materials, including propane raw materials, catalyst raw materials, and the like.

The pre-rising system is used to mix homogeneously the reaction raw materials and deliver the reaction raw materials to the reaction system. In this example, the pre-rising system is the pre-riser 4. The pre-riser 4 has a cylindrical structure, in which the inlet of the pre-riser 4 is provided with a ring distributor 2, and the outlet of the pre-riser 4 is a tapered and constricted neck structure with a thin top and a thick bottom.

The reaction system is used for catalytic dehydrogenation reaction of propane. The reaction system includes a turbulent bed reactor 9 and a conical riser 5, wherein:

The conical riser 5 is in the shape of a conical structure with a thick top and a thin bottom. The ratio of the outlet diameter to the inlet diameter of the conical riser 5 is less than or equal to 3 and greater than 1. The angle between the wall generatrix and the central vertical line of the conical riser 5 is between 0° and 10°.

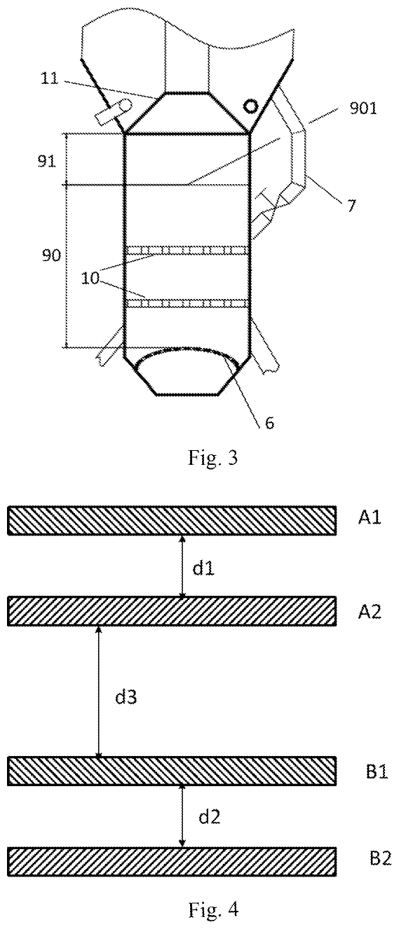

As shown in FIG. 3, a first perforated distribution plate 6 and one or more groups of cross-flow grids 10 are arranged inside the turbulent bed reactor 9. Inside the turbulent bed reactor 9, there is generally a catalyst bed 90 formed by catalyst particles above the upper surface of the first perforated distribution plate 6, and the height of the catalyst bed 90 is 2-m. When the catalytic dehydrogenation reaction of propane is carried out, the catalyst on the upper surface 901 of the catalyst bed is in a fluctuating state, and a splash zone 91 is formed above the catalyst bed, and the height of the splash zone 91 is 1500 mm-60000 mm.

The inlet section of the turbulent bed reactor 9 is an expanded neck structure with a thin bottom and a thick top, and the first perforated distribution plate 6 is arranged on the upper part of the inlet section. The aperture of the first perforated distribution plate is preferably Φ100 mm-Φ250 mm.

The cross-flow grids 10 are composed of multiple groups of grids arranged in layers in the vertical direction. Each group includes two layers of grids, as shown in FIG. 4, and the vertical distances d1 and d2 between the two layers of grids in the same group are greater than or equal to 300 mm, and the vertical distance d3 between two adjacent groups of grids is 500 mm to 4000 mm; wherein the lowermost layer of grid is located above the first perforated distribution plate 6, and the vertical distance therebetween is greater than or equal to 500 mm. The uppermost layer of grid is immersed in the catalyst bed 90, and the vertical distance between the uppermost layer of grid and the upper surface 901 of the catalyst bed is more than or equal to 500 mm. The openings of each layer of grids can be rectangular, square or the like, and the side length of the openings in each layer of grids is generally 100 mm-500 mm.

The gas-solid separation system is used to separate the gas and solid phases discharged from the reaction system and collect the dense phase catalyst. The gas-solid separation system includes a casing, a gas collection hood 11, a dilute phase pipe 13, a low-wear gas-solid separation device and a secondary cyclone separator 16, wherein the low-wear gas-solid separation device is a cantilever type gas-solid fast separator 14. The gas collection hood 11, the dilute phase pipe 13, the cantilever type gas-solid fast separator 14, and the secondary cyclone separator 16 are arranged coaxially inside the casing of the gas-solid separation system from bottom to top.

The top of the casing of the gas-solid separation system is provided with a product gas outlet 17.

The gas collection hood 11 is tapered with a thin top and a thick bottom, which is conducive to the rapid collection of materials. The angle between the generatrix of the gas collection hood and the central axis is 30°-70°. The bottom end of the gas collection hood 11 is located above the splash zone 91 in the reaction system. The distance between the bottom end of the gas collection hood 11 and the upper surface 901 of the catalyst bed is 1500 mm-6000 mm.

The top of the dilute phase pipe 13 is closed, and the lateral side is provided with a square outlet or a rectangular outlet. The outlets on both lateral sides of the dilute phase pipe 13 are arranged symmetrically along the central axis of the dilute phase pipe 13.

The cantilever type gas-solid fast separator 14 includes a cover 15 and two or more cantilevers. Herein, the cover 15 is in a cylindrical shape with an opening at the bottom. An outlet pipe 35 is provided on the top of the cover 15, and the outlet pipe 35 is connected to the inlet of the secondary cyclone separator 16.

The cantilever is located inside the cover 15, and the cantilever is in one-to-one correspondence with the outlets on the side of the dilute phase pipe 13. The extension direction of the cantilever is horizontal extension or downward spiral extension. The radial distance between the end of the cantilever and the inner wall of the cover 15 is relatively short, generally within 500 mm, so that the catalyst separated by the cantilever can quickly settle down along the inner wall of the cover 15 after it is discharged, and the settled catalyst piles up at the bottom of the casing of the gas-solid separation system to form a dense phase bed.

The bottom of the annular space between the cover 15 and the casing of the gas-solid separation system is provided with a ring distributor 12, which is located below the bottom opening of the cover 15, and used to provide fluidization power for the catalyst in the dense phase bed.

In this example, the internal circulation pipeline specifically includes a first catalyst circulation pipeline 7 and a second catalyst circulation pipeline 34. Among them, the first catalyst circulation line 7 is used to deliver the dense phase catalyst collected in the gas-solid separation system to the turbulent bed reactor 9, and the second catalyst circulation line 34 is used to deliver the catalyst with low coke yield collected in the turbulent bed reactor 9 to the raw material delivery pipeline 1.

The inlet of the first catalyst circulation line 7 is connected to the casing of the gas-solid separation system, and the connection is located above the ring pipe distributor 12; the outlet of the first catalyst circulation line 7 is connected to the turbulent bed reactor 9, and the connection is located above the uppermost layer of grid in the cross-flow grids 10.

The inlet of the second catalyst circulation line 34 is connected to the turbulent bed reactor 9, and the connection therebetween is located between the lowermost layer of grid in the cross-flow grids 10 and the first perforated distribution plate 6, and the outlet of the second catalyst circulation line 34 is connected to the inlet of the raw material delivery pipeline 1.

The pre-riser 4, the conical riser 5, the turbulent bed reactor 9, the gas collection hood 11, the dilute phase pipe 13, the cantilever type separator 14, and the secondary cyclone separator 16 are generally arranged coaxially.

In the above fluidized bed reactor, the connection relationship of the systems is as follows: the raw material delivery pipeline 1 runs through the inlet of the pre-riser 4 from bottom to top and extends into the inside of the pre-riser 4, and the outlet of the raw material delivery pipeline 1 is located above the ring distributor 2; the outlet of the pre-riser 4 is connected to the inlet of the conical riser 5; the outlet of the conical riser 5 is connected to the inlet of the turbulent bed reactor 9; the outlet of the turbulent bed reactor 9 is connected to the inlet of the gas collection hood 11; the outlet of the gas collection hood 11 is connected to the inlet of the dilute phase pipe 13; the outlet on the lateral side of the dilute phase pipe 13 is connected to the inlet of the cantilever type gas-solid fast separator 14; the outlet of the cantilever in the cantilever type gas-solid fast separator 14 is connected to the solid outlet and the gas outlet of the cover 15; the gas outlet of the cover 15 is connected to the inlet of the secondary cyclone separator 16.

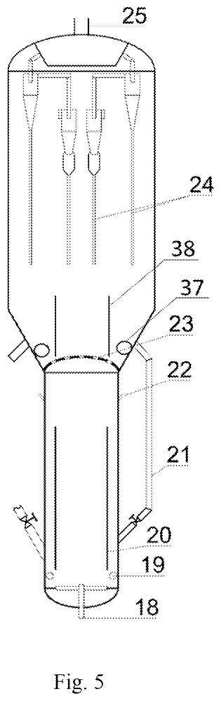

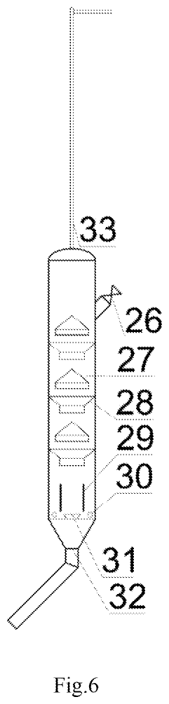

As shown in FIGS. 1, 5 and 6, the gas-solid airlift loop regenerator includes a first regeneration system, a second regeneration system, and a first stripper communicating in this order. The first regeneration system is located below the second regeneration system. The first regeneration system is used to carry out the first coke-burning regeneration on the catalyst. The first regeneration system includes a first casing, a main air distributor 18, a first ring pipe distributor 19 and a first draft tube 20.

The main air distributor 18, the first ring pipe distributor 19 and the first draft tube 20 are located inside the first casing. The first draft tube 20 is located in the middle of the first casing, and the height of the first draft tube 20 is 1 m-5 m. The main air distributor 18 is located below the vertical projection area of the first draft tube 20, and the vertical distance between the main air distributor 18 and the first draft tube 20 is greater than or equal to 300 mm. The first ring pipe distributor 19 is arranged in the vertical projection area between the first draft tube 20 and the first casing, and the vertical distance between the top of the first ring pipe distributor 19 and the bottom end of the first draft tube 20 is less than or equal to 500 mm. Thus, the interior of the first regeneration system is divided by the first draft tube into the inner space of the draft tube and the annulus area between the first draft tube and the first casing. The lower part of the first casing is provided with an inlet, the top of the first casing is provided with an outlet, and a fuel feed nozzle 22 is provided between the outlet of the first casing and the first draft tube 20, and the fuel feed nozzle 22 is connected to the internal space of the first casing, and the vertical distance between the fuel feed nozzle 22 and the first draft tube 20 is 200 mm-1500 mm. In this example, there are multiple fuel feed nozzles 22, and the outlets of the multiple fuel feed nozzles 22 are evenly distributed along the circumferential direction of the first casing.

The second regeneration system is used to carry out a further coke-burning regeneration on the catalyst. The second regeneration system includes a second casing, a second perforated distribution plate 23, a second ring pipe distributor 37, a second draft tube 38 and a combined cyclone separator 24. Among them, the second perforated distribution plate 23, the second ring pipe distributor 37, the second draft tube 38, and the combined cyclone separator 24 are located inside the second casing. The bottom of the second casing is provided with an inlet having a thick top and a thin bottom, the top of the second casing is provided with a gas outlet 25, and the bottom of the second casing is provided with a solid outlet. The second perforated distribution plate 23 is arranged above the inlet of the second casing, the second draft tube 38 is arranged above the second perforated distribution plate 23, and the second ring pipe distributor 37 is provided in the vertical projection area between the second draft tube 38 and the second casing. The height of the second draft tube 38 is 1 m-3 m, and the vertical distance between the lower end of the second draft tube 38 and the second ring pipe distributor 37 is less than or equal to 500 mm.

A regenerant circulation pipeline 21 is connected between the bottom of the second regeneration system and the bottom of the first regeneration system. The regenerant circulation pipeline 21 is used to balance the accumulation height of the catalyst in the first regeneration system and the second regeneration system.

The first stripper is used to separate the catalyst entrained in the oxygen-containing flue gas.

The first stripper includes a third casing, a partition member, a third draft tube 29, a ring pipe steam distributor 30 and a stripping steam distributor 31. A ring baffle 27, a disk baffle 28, the third draft tube 29, and the stripping steam distributor 31 are arranged in this order from top to bottom inside the third casing. The ring pipe steam distributor 30 is arranged in the vertical projection area between the third casing and the third draft tube 29. The first stripper is respectively provided with an oil and gas outlet pipeline 33, an inlet 26 and a solid outlet 32 from top to bottom.

The outlet of the first regeneration system is connected to the inlet of the second regeneration system, and the solid outlet of the second regeneration system is connected to the inlet 26 of the first stripper. The oil and gas outlet pipeline 33 of the first stripper is connected to the inner space of the second regeneration system.

The fluidized bed reactor and the gas-solid airlift loop regenerator are arranged in parallel in the horizontal direction, connected by the recirculation inclined pipe 8 and the regeneration inclined pipe 3. Specifically, the inlet of the recirculation inclined pipe 8 is connected to the turbulent bed reactor 9 in the fluidized bed reactor, and the connection between the recirculation inclined pipe 8 and the turbulent bed reactor 9 is located above the connection between the first catalyst circulation pipeline 7 and the turbulent bed reactor 9; the outlet of the recirculation inclined pipe 8 is connected to the inlet of the first regeneration system. The inlet of the regeneration inclined pipe 3 is connected to the solid outlet 32 of the first stripper, the outlet of the regeneration inclined pipe 3 is connected to the inlet of the pre-riser 4, and the connection between the regeneration inclined pipe 3 and the pre-riser 4 is not below the outlet of the raw material delivery pipeline 1.

In some specific embodiments, a second stripper may be further connected between the recirculation inclined pipe 8 and the first regeneration system to replace the propylene product gas adsorbed in the spent catalyst.

### Example 2

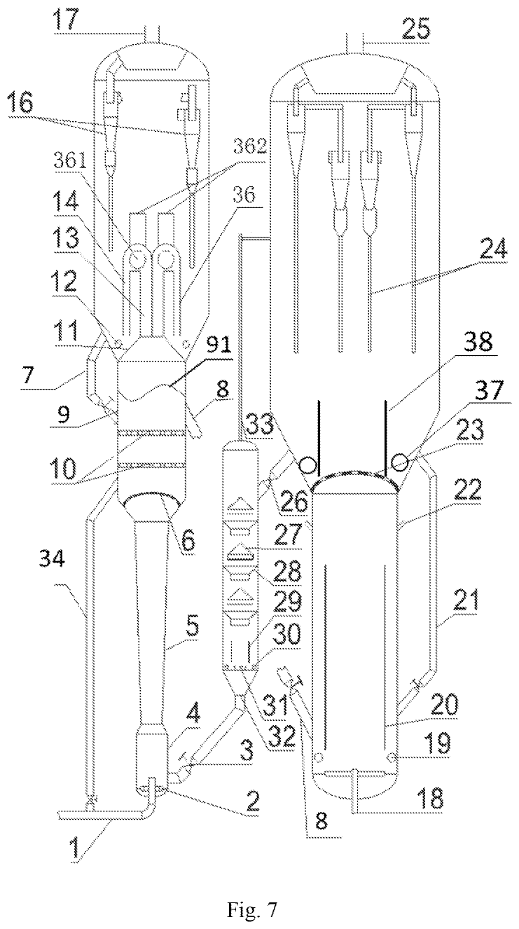

This example provides a coupled fluidized beds reactor-regenerator apparatus for catalytic dehydrogenation of propane. As shown in FIG. 7, the coupled apparatus is similar in structure to the coupled apparatus in Example 1, only except that in this example, the cantilever type gas-solid fast separator 14 in Example 1 is replaced with an ultra-short horizontal fast separator 36 as a low-wear gas-solid separation device. The use of the ultra-short horizontal fast separator 36 can further reduce the contact time of the gas and solid phases and reduce the secondary reaction.

In this example, the outlet of the dilute phase pipe 13 is arranged at the top, and the top outlet is connected to the central pipe 361 of the ultra-short horizontal fast separator, and the solid outlet of the central pipe 361 of the ultra-short horizontal fast separator is downward. The outlet of the gas phase outlet pipe 362 of the ultra-short horizontal fast separator is located above the gas collection hood 11.

### Example 3

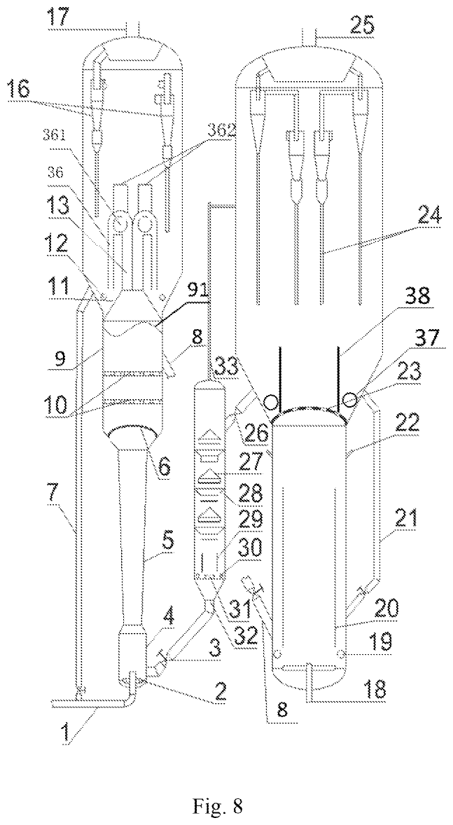

This example provides a coupled fluidized beds reactor-regenerator apparatus for catalytic dehydrogenation of propane. As shown in FIG. 8, the coupled apparatus is similar in structure to the coupled apparatus in Example 2, only except that in this example, the gas collection hood 11 is partially immersed in the splash zone 91, and meanwhile the second catalyst circulation pipeline is omitted in this example, but the inlet of the first catalyst circulation pipeline 7 is connected to the bottom of the casing of the gas-solid separation system and the outlet of the first catalyst circulation pipeline 7 is connected to the raw material delivery pipeline 1.

### Example 4

This example provides a process for catalytic dehydrogenation of propane, which is carried out in the coupled apparatus of Example 1. The process comprises the following steps.

The propane and catalyst are delivered to the pre-riser 4 through the raw material delivery pipeline 1, and then enter the conical riser 5 for rising movement and catalytic dehydrogenation reaction of propane to produce gaseous products such as propylene.

In the conical riser 5, the product gas (propylene) and unreacted raw material gas (propane) entrain catalyst particles and rise along the conical riser 5 together. In the conical riser 5, the catalytic dehydrogenation of propane is carried out at a reaction temperature of 600-680° C., a reaction pressure of less than or equal to 1 MPa, for a reaction time of 5 s-15 s. The gas and solid phases composed of catalyst particles, raw material gas and product gas output from the conical riser 5 enter the turbulent bed reactor 9 through the first perforated distribution plate 6 at a uniform speed, and continuously react for catalytic dehydrogenation and move upward.

The catalyst with a low coke yield between the first perforated distribution plate 6 and the lowermost layer of grid enters the second catalyst circulation pipeline 34 and then returns to the raw material delivery pipeline 1, and then enters the pre-riser 4 again to participate in the reaction process. The rest of the catalyst is entrained by the gas and continues to move upward. Under the action of the cross-flow grids 10, bubbles generated during rising of the gas and solid phases are broken, and back-mixing is suppressed. In the turbulent bed reactor 9, catalytic dehydrogenation of propane is carried out at a reaction temperature of 500-620° C. and a reaction pressure of less than or equal to 1 MPa for a reaction time of 5 s-15 s. When the gas and solid phases reaches the uppermost layer of grid of the cross-flow grids 10, most of the catalyst with a relatively high coke yield that needs to be regenerated in the turbulent bed reactor 9 enters the recirculation inclined pipe 8, and the catalyst entering the recirculation inclined pipe is the spent catalyst. The remaining and small amount of catalyst (about 10% of the total amount of catalyst) is entrained by the gas and moves upwards into the gas collection hood 11.

The gas entraining the catalyst enters the dilute phase pipe 13 quickly after being collected by the gas collection hood 11, and then enters the cantilever type gas-solid fast separator 14 through the dilute phase pipe 13 for gas-solid separation, wherein the separated solid catalyst escaping from the cantilever is blocked by the inner wall of the cover 15, falls to the bottom of the cover 15, and accumulates to form a dense phase bed. The gas separated by the cantilever type gas-solid fast separator 14 moves upward inside the cover 15, and enters the secondary cyclone separator 16 through the outlet pipe 35 at the top of the cover for further gas-solid separation. The gas separated by the secondary cyclone separator 16 is discharged and collected by the product gas outlet 17, while the solid catalyst separated by the secondary cyclone separator 16 falls to the dense phase bed, and under the blowing effect of the ring pipe distributor 12, the catalyst in the dense phase bed is delivered back to the turbulent bed reactor 9 by the first catalyst circulation pipeline 7, and then enters the recirculation inclined pipe 8.

The spent catalyst in the recirculation inclined pipe 8 then enters the first regeneration system, and under the blowing action of the main air distributor 18 and the first ring pipe distributor 19, the spent catalyst moves upward in the draft tube and when it reaches the fuel feed nozzle 22, the temperature rises under the action of the fuel. The heated spent catalyst enters downward into the annulus area between the first draft tube 20 and the first ring pipe distributor 19, and then moves radially into the draft tube again for circulating flow, so that the temperature in the regeneration system is maintained at the temperature required for the coke-burning reaction. During the circumfluence movement of the spent catalyst, the high-temperature spent catalyst that is in contact with the fuel is quickly mixed with the low-temperature spent catalyst that is not in contact with the fuel, so that the whole spent catalyst in the first regeneration system is maintained at a relatively high temperature (620-750° C.) and the temperature is evenly distributed to achieve efficient and stable coke-burning regeneration. In a specific embodiment, the coke-burning temperature of the spent catalyst in the first regeneration system can be controlled by regulating the temperature and flow rate of the main air distributor 18 and the flow rate of the fuel. Generally speaking, the greater the gas flow rate of the main air distributor 18 and the greater the flow rate of the fuel, the more sufficient the coke-burning degree of the spent catalyst is.

After the first coke-burning regeneration, the spent catalyst enters the second regeneration system along with the entrainment of the flue gas. Inside the second regeneration system, the spent catalyst first passes through the second perforated distribution plate 23 to achieve uniform distribution in the inner space, and then it is circulated inside the second draft tube 38 and in the annular area between the second draft tube 38 and the second casing. During the flow process, the high-temperature spent catalyst is subjected to the second coke-burning regeneration, and the spent catalyst is regenerated through the continuous coke-burning regeneration process, recovers its catalytic activity, and is converted into a regenerant. Then the regenerant is entrained by the flue gas and moves upwards, and enters the combined cyclone separator 24 for oil and gas separation. The separated flue gas is discharged from the outlet 25, and the separated spent catalyst enters the first stripper through the solid outlet of the second casing and the inlet 26 in turn.

The spent catalyst passes through the ring baffle 27 and the disc baffle 28 successively and enters the third draft tube 29 in the first stripper, and is circulated under the action of the ring pipe steam distributor 30 and the stripping steam distributor 31. The oxygen-containing flue gas is removed during the movement, and then the regenerant returns to the pre-riser 4 through the solid outlet 32 of the first stripper to participate in the catalytic dehydrogenation reaction of propane again.

In the above process for catalytic dehydrogenation of propane, the raw material propane and catalyst enter the pre-riser 4 from the bottom through the delivery pipe 1, and the regenerant particles enter the middle part of the pre-riser 4 through the regeneration inclined pipe 3. When two streams of materials are fast mixed in the pre-riser 4, it enters the conical riser 5 from the outlet of the pre-riser 4, and flows upward along the axis of the conical riser while the catalytic dehydrogenation reaction of propane proceeds. Since the catalytic dehydrogenation reaction of propane is a molecule increased reaction, new gas is continuously generated, and the gas flow is getting faster and faster. The shape of thick top and thin bottom of the conical riser 5 can maintain the speed of the gas and solid phases unchanged substantially, so as to ensure an advection flow while reducing the catalyst wear.

The advection flow can eliminate the back-mixing of the gas and solid phases, improve the selectivity of the reaction without additional rising media, and reduces the load and energy consumption of the device. The inlet section of the turbulent bed reactor 9 is a expanded neck structure, which can be well connected with the outlet of the conical riser 5, and the first perforated distribution plate 6 with a large diameter can be provided at the inlet section of the turbulent bed reactor 9, which is favorable for uniform distribution of the catalyst particles after entering the turbulent bed reactor 9. In the turbulent bed reactor 9, the gas and solid phases continue to react, thereby further prolonging the reaction time, and the first catalyst pipeline 7 continuously deliver the dense phase catalyst collected by the gas-solid separation system to the turbulent bed reactor 9 to ensure a high catalyst-to-oil ratio. The cross-flow grids 10 arranged above the first perforated distribution plate 6 can break the bubbles generated during rising of the gas and solid phases, increase the specific surface area of the gas-solid contact (that is, the contact area between the gas phase and the solid phase in a unit volume), and greatly reduce the back-mixing phenomenon in the turbulent bed reactor 9 and reduce the secondary reaction.

During the catalytic dehydrogenation of propane, the catalyst with low coke yield at the bottom of the turbulent bed reactor 9 enters the second catalyst circulation pipeline 34, and then merges into the raw material delivery pipeline 1, and is again delivered to the conical riser 5 through the pre-riser 4 to participate in the reaction. In the present disclosure, by adopting the design of the internal circulation pipeline, the catalyst with a low coke yield in the turbulent bed reactor 9 is delivered again to the conical riser 5, which can greatly supplement and increase the catalyst concentration in the conical riser 5, and meanwhile adjust the amount of catalyst involved in the reaction flexibly in time.

At the uppermost layer of grid of the cross-flow grids 10 in the turbulent bed reactor 9, most of the catalyst with a relatively high coke yield is used as the spent catalyst and enters the gas-solid airlift loop regenerator from the recirculation inclined pipe 8 for coke-burning regeneration.

The gas coming out of the turbulent bed reactor 9 entrains the remaining small amount of catalyst particles into the gas collection hood 11. Because the gas collection hood 11 is closer to the catalyst bed 90 in the turbulent bed reactor 9 but higher than the splash zone 91, the gas and solid phases discharged from the turbulent bed reactor 9 can quickly enter the gas-solid separation system through the gas collection hood 11 for gas-solid separation, which greatly shortens the further contact time between the gas and the catalyst in addition to the time required for the propane dehydrogenation reaction, and reduces the degree of secondary reaction. The gas and solid phases entering the gas-solid separation system first passes through the dilute phase pipe 13 and enters the cantilever type gas-solid fast separator 14 for the first gas-solid separation. A mixture of the catalyst and the gas is drawn out from multiple cantilevers of the cantilever type gas-solid fast separator 14. The distance between the outlet of the cantilever and the wall of the cover 15 is short, and the catalyst particles coming out of the cantilever only need to move a small radial distance to quickly reach the inner wall of the cover 15, so that efficient separation can be achieved at a low tangential velocity, and the gas-solid separation efficiency can reach 98% or more, while greatly reducing the pressure drop in the separation. The catalyst particles fall along the cover 15, and form a dense phase bed at the lower part of the cover 15. The dense phase bed forms a material seal to prevent gaseous products from flowing out from the lower end of the cover 15. The separated gaseous product moves upward along the cover 15, and enters the settling section (that is, the outer cylinder part of the cantilever type separator 14) from the outlet pipe 35 at the top of the cover, and then enters the secondary cyclone separator 16. Since about 98% of the catalyst particles are separated from the cantilever type gas-solid fast separator 14, the amount of particles entering the secondary cyclone separator 16 is low, which greatly avoids the wear of a large amount of catalyst in the secondary cyclone separator 16. The catalyst particles separated from the secondary cyclone separator 16 fall back into the dense phase bed at the lower end of the cover 15 along the dipleg. The catalyst in the dense phase bed falls into the turbulent bed reactor 9 along the first catalyst circulation pipeline 7 under the blowing effect of the ring pipe distributor 12. The gas separated from the secondary cyclone separator 16 is discharged from the gas-solid separation system along the product gas outlet 17.

The recirculation inclined pipe 8 first delivers the spent catalyst collected from the turbulent bed reactor 9 to the first regeneration system. Since the amount of gas introduced into the inner area of the first draft tube 20 is greater than the amount of gas introduced into the annulus area, the density of the spent catalyst in the inner area of the first draft tube 20 is smaller than that in the annulus area, so that the pressure in the an annulus area is greater than the pressure in the inner area of the first draft tube 20. The pressure difference pushes the spent catalyst particles to flow upward in the first draft tube 20 and downward in the annulus area. The fuel is injected into the fuel feed nozzle 22 above the first draft tube 20. Due to the significant radial flow near the fuel feed nozzle 22, the high-temperature catalyst and the surrounding low-temperature catalyst can be quickly mixed to ensure the uniform distribution of temperature in the first regeneration system and achieve a stable coke-burning regeneration. The spent catalyst after the first coke-burning is entrained by the flue gas and enters the second regeneration system through the second large-hole distribution plate 23. It is circulated along the inner space of the second draft tube 38 and the annulus area between the second draft tube 38 and the second casing, and undergoes the second coke-burning reaction during the movement. After two coke-burning, the spent catalyst has completely recovered its activity and converted to a regenerant. The regenerant enters the stripper, and is stripped countercurrently by the first stripper to remove the oxygen-containing flue gas adsorbed among the solid particles, and then the regenerant is delivered back to the pre-riser 4, and again participates in the catalytic dehydrogenation reaction of propane as a catalyst. The flue gas discharged from the first stripper is delivered to the combined cyclone separator 24 through the oil and gas outlet pipeline 33 for gas-solid separation, so as to separate the regenerant particles entrained in the flue gas.

The processes of catalytic dehydrogenation of propane carried out in Example 2 and Example 3 are similar to the above, only except:
as compared with Example 1, in the process carried out by the coupled apparatus in Example 2, the gas and solid phases output from the turbulent bed reactor 9 enters the ultra-short horizontal fast separator 36 through the gas collection hood 11 and the dilute phase pipe 13 for rapid gas-solid separation;
as compared with Example 2, in the process carried out by the coupled apparatus in Example 3, the catalyst in the dense phase bed at the bottom of the gas-solid separation system is directly delivered to the raw material delivery pipeline 1 through the first catalyst circulation pipeline 7.

In practical production, the propylene yield in the above process for catalytic dehydrogenation of propane can reach 37%-40%, the single-pass conversion rate can reach 42%-47%, and the selectivity can reach 90% or more, with high production efficiency and yield.

### Example 5

This example provides a coupled fluidized beds reactor-regenerator apparatus for catalytic dehydrogenation of propane. This apparatus is similar in structure to the coupled apparatus in Example 1, only except that the product gas outlet 17 of the fluidized bed reactor in this example is further connected with a product gas separation system.

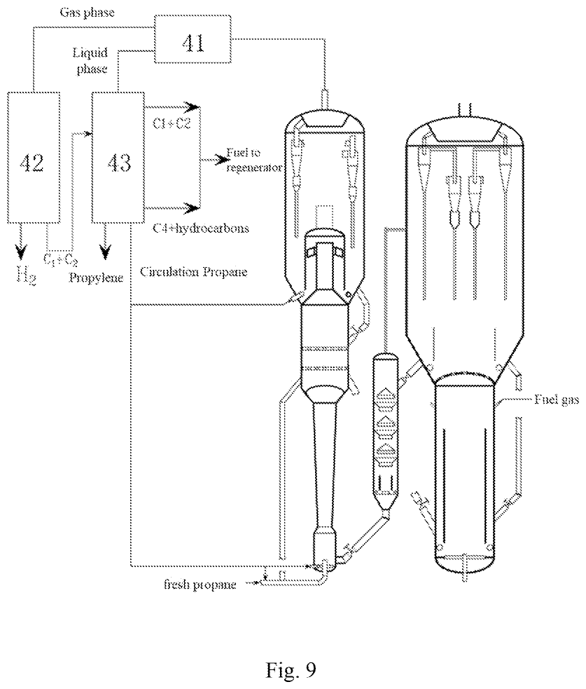

As shown in FIG. 9, the product gas separation system includes a compression condensing unit 41, a first separation unit 42 and a second separation unit 43.

The compression condensing unit 41 is used to carry out stagewise compression condensation and gas-liquid separation on the gas, and includes multi-stage compressors, inter-stage cooling equipment and inter-stage separation tanks arranged between the multi-stage compressors. The multi-stage compressors are used to compress the gas sequentially, the inter-stage cooling equipment is used to reduce the gas temperature, and the inter-stage separation tank is used to separate the condensed liquid phase. The compression condensing unit 41 is provided with an inlet, a gas phase outlet and a liquid phase outlet. Among them, the inlet of the compression condensing unit 41 is connected to the product gas outlet 17, and the gas phase outlet of the compression condensing unit 41 is connected to the inlet of the first separation unit 42, and the liquid phase outlet of the compression condensing unit 41 is connected to the inlet of the second separation unit 43.

The first separation unit 42 is used to separate hydrogen and light hydrocarbons (C1, C2 and liquid phase impurities) in the gas phase. The first separation unit 42 is provided with a hydrogen outlet and a light hydrocarbon outlet, and the light hydrocarbon outlet of the first separation unit 42 is connected to the inlet of the second separation unit 43.

The second separation unit 43 is provided with a plurality of depropanizers, and the separation of light hydrocarbons, propylene, propane and C4+ heavier hydrocarbons can be achieved through a rectification process. The second separation unit 43 is provided with a propylene outlet, a propane outlet and a fuel hydrocarbon outlet. The fuel hydrocarbon outlet of the second separation unit 43 is connected to the fuel feed nozzle 22 in the gas-solid airlift loop regenerator, for delivering the separated light hydrocarbons and C4+ to the first regeneration system as fuel. The propane outlet of the second separation unit 43 is respectively connected to the ring pipe distributor 12, the ring distributor 2 and the raw material delivery pipeline 1. The propane entering the ring pipe distributor 12 and the ring distributor 2 as a fluidizing gas can promote the flow of the catalyst particles, and the propane entering the raw material delivery pipeline 1 is used as the raw material gas together with fresh propane to participate in the catalytic dehydrogenation reaction of propane.

### Example 6

This example provides a process for catalytic dehydrogenation of propane, which is carried out in the coupled apparatus of Example 5. The process includes all the steps of the process of Example 4, and further includes the following steps.

The gas discharged from the product gas outlet 17 first enters the compression condensing unit 41 for stagewise compression condensation and gas-liquid separation, and the separated gas phase (containing hydrogen and light hydrocarbons) enters the first separation unit 42, and the separated liquid phase (containing propane, propylene and C4+) enters the second separation unit 43.

The gas phase is subjected to gas separation in the first separation unit, and the separated hydrogen is used as a product gas and discharged from the device; the separated light hydrocarbons enter the second separation unit 43.

The second separation unit 43 separates propylene, propane, light hydrocarbons and C4+ respectively by rectification technology, wherein: propylene is used as a product gas and discharged from the device, the light hydrocarbons and C4+ are introduced into the fuel feed nozzle 22 as fuel after decompression, and propane is divided into three parts, wherein one enters the ring pipe distributor 12 as the fluidizing gas, another one enters the ring distributor 2 as the fluidizing gas, and the remaining one enters the raw material delivery pipeline 1 to be mixed with fresh propane as the reaction raw material.

### Example 7

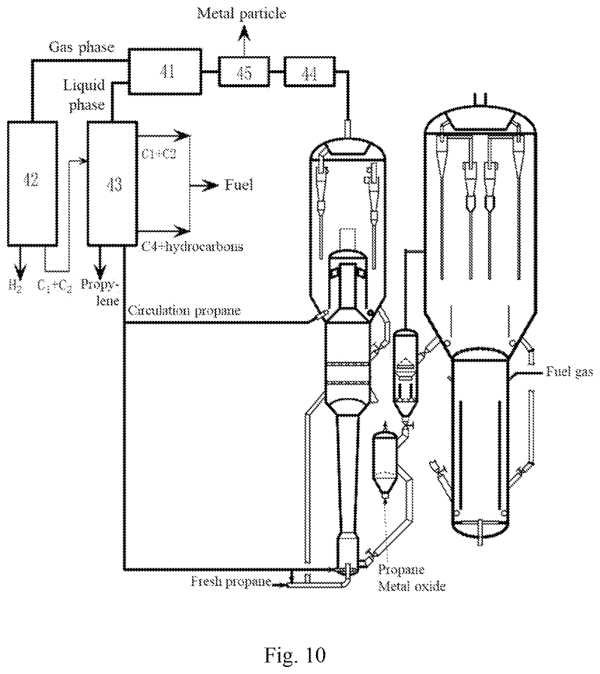

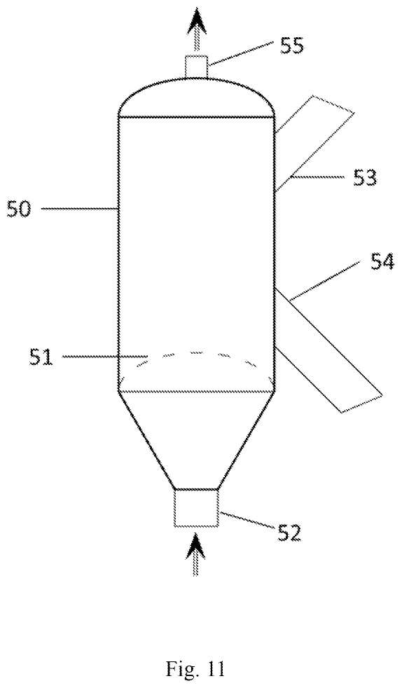

This example provides a coupled fluidized beds reactor-regenerator apparatus for catalytic dehydrogenation of propane. As shown in FIG. 10, this apparatus is similar in structure to the coupled apparatus in Example 5, only except for the following differences.

In this example, the product gas separation system further includes a primary condensation unit 44 provided with an inlet and an outlet, and a gas-solid separation unit 45 provided with an inlet, a solid outlet and a gas outlet.

The inlet of the primary condensation unit 44 is connected to the product gas outlet 17, and the outlet of the primary condensation unit 44 is connected to the inlet of the gas-solid separation unit 45. The gas outlet of the gas-solid separation unit 45 is connected to the inlet of the compression condensing unit.

The gas-solid airlift loop regenerator in this example further includes a catalyst activator 50 for supplementing the regenerant with a metal active component. As shown in FIG. 11, the inner bottom of the catalyst activator 50 is provided with a third perforated distribution plate 51. The catalyst activator 50 is provided with a raw material inlet 52, a solid outlet 54, a regenerant inlet 53 and a gas outlet 55 in this order from bottom to top. Catalyst Activator 50. The raw material inlet 52 is used to receive the raw materials (generally propane gas and a metal oxide) for forming the metal active component, the regenerant inlet 53 is connected to the solid outlet 32 of the first stripper, the solid outlet 54 is connected to the regeneration inclined pipe 3, and the gas outlet 55 is in communication with the gas-solid separation system, specifically, it may be connected to the ring pipe distributor 12.

### Example 8

This example provides a process for catalytic dehydrogenation of propane, which is carried out in the coupled apparatus of Example 7. The process includes all the steps of the process of Example 4, and further includes the following steps.

The regenerant output from the first stripper enters the catalyst activator 50 through the regenerant inlet 53, and the metal oxide in the catalyst activator 50 is reduced by propane to a metal elementary substance. The metal elementary substance is deposited in the regenerant to form the metal active component, so that the catalytic activity of the regenerant is improved, and the regenerant supplemented with the metal active component enters the regeneration inclined pipe through the solid outlet 54. The gas in the catalyst activator 50 enters the gas-solid separation system through the gas outlet 55 for gas-solid separation. The catalyst particles separated from the secondary cyclone separator 16 settle to the dense phase bed, and the gas separated by the secondary cyclone separator 16 enters the product gas separation system through the product gas outlet 17.

The gas discharged from the product gas outlet 17 first enters the primary condensation unit 44 for condensation, and the metal vapor in the gas is condensed into metal particles, and then the gas entrained with the metal particles enters the gas-solid separation unit 45 to separate the metal particles from the gas. The separated metal particles are discharged from the product gas separation system, and sent to the catalyst activator 50 as a raw material after oxidization. The separated gas enters the compression condensing unit 41.

In the compression condensing unit 41, the gas is subjected stagewise compression condensation and gas-liquid separation. The separated gas phase (containing hydrogen and light hydrocarbons) enters the first separation unit 42, and the separated liquid phase (containing propane, propylene and C4+) enters the second separation unit 43.

The gas phase is subjected to gas separation in the first separation unit, and the separated hydrogen is used as a product gas and discharged from the device; the separated light hydrocarbons enter the second separation unit 43.

the second separation unit 43 separates propylene, propane, light hydrocarbons and C4+ respectively by rectification technology, wherein: propylene is used as a product gas and discharged from the device, the light hydrocarbons and C4+ are introduced into the fuel feed nozzle 22 as fuel after decompression, and propane is divided into four parts, wherein one enters the ring pipe distributor 12 as the fluidizing gas, another one enters the ring distributor 2 as the fluidizing gas, a further one enters the raw material delivery pipeline 1 to be mixed with fresh propane as the reaction raw material, and the remaining one enters the catalyst activator 50 as the raw material.

As compared with the process of Example 4, the process of this example includes a procedure of supplementing the catalyst with the active component after the catalyst is regenerated, which can further improve the catalyst activity, so that the propylene yield, single-pass conversion rate and selectivity of the catalytic dehydrogenation of propane process are significantly improved as compared with the effect of Example 4.

1. A fluidized bed reactor comprising a raw material delivery system, a pre-rising system, a reaction system, a gas-solid separation system, and an internal circulation pipeline, wherein:
the reaction system comprises a conical riser and a turbulent bed reactor, and the cross-sectional diameter of the conical riser gradually increases from an inlet to an outlet;
the raw material delivery system, the pre-rising system, the conical riser, the turbulent bed reactor, and the gas-solid separation system are consecutively connected in this order from bottom to top, wherein the bottom of the gas-solid separation system is provided with an outlet connected to an inlet of the internal circulation pipeline, and an outlet of the internal circulation pipeline is connected to the raw material delivery system and/or the reaction system; and
the gas-solid separation system is provided with a product gas outlet.

2. The fluidized bed reactor according to claim 1, wherein the angle between the wall generatrix of the conical riser and the central vertical line of the conical riser is between 0° and 10°, and wherein the ratio of the outlet diameter to the inlet diameter of the conical riser is less than or equal to 3 and greater than 1.

3. The fluidized bed reactor according to claim 1, wherein:
the bottom of the turbulent bed reactor is provided with a first perforated distribution plate;
the turbulent bed reactor is further provided with one or more layers of grids located above the first perforated distribution plate and arranged in layers along the vertical direction; and
a catalyst bed is formed in the inner space of the turbulent bed reactor, and at least the uppermost layer of grid is located inside the catalyst bed.

4. The fluidized bed reactor according to claim 1, wherein the vertical distance between the lowermost layer of grid and the first perforated distribution plate is greater than or equal to 500 mm.

5. The fluidized bed reactor according to claim 1, wherein the grids are one or more groups of cross-flow grids including two layers of grids in each group, wherein the vertical distance between the two layers of grids in the same group is greater than or equal to 300 mm, and wherein the vertical distance between two adjacent groups of grids is 500 mm to 4000 mm.

6. The fluidized bed reactor according to claim 1, wherein the gas-solid separation system comprises a casing, wherein a gas collection hood, a dilute phase pipe, a low-wear gas-solid separation device and a cyclone separator are provided inside the casing, wherein an inlet of the gas collection hood is connected to an outlet of the turbulent bed reactor, and wherein the gas collection hood, the dilute phase pipe, the low-wear gas-solid separation device and the cyclone separator are consecutively connected in this order.

7. The fluidized bed reactor according to claim 6, wherein the gas collection hood is located above the catalyst bed, and wherein the vertical distance between the gas collection hood and the upper surface of the catalyst bed is 1500 mm to 6000 mm.

8. The fluidized bed reactor according to claim 6, wherein:
the dilute phase pipe is located above the gas collection hood;
the low-wear gas-solid separation device is provided with a solid outlet downward opened and a gas outlet upward opened;
the gas outlet of the low-wear gas-solid separation device is connected to an inlet of the cyclone separator; and
a gas outlet of the cyclone separator is connected to the product gas outlet.

9. The fluidized bed reactor according to claim 6, wherein the low-wear gas-solid separation device comprises a cantilever type gas-solid fast separator or an ultra-short fast separator,
wherein the cantilever type gas-solid fast separator comprises a cover and a cantilever located inside the cover, an inlet of the cantilever is connected to an outlet of the dilute phase pipe, an end of the cantilever is provided with a solid outlet, the bottom of the cover is open, and the top of the cover is provided with a gas outlet.

10. The fluidized bed reactor according to claim 1, wherein the internal circulation line is a first catalyst circulation line having an inlet connected with the gas-solid separation system and an outlet connected with the raw material delivery system.

11. The fluidized bed reactor according to claim 1, wherein the internal circulation pipeline comprises a first catalyst circulation pipeline having an inlet connected with the gas-solid separation system and an outlet connected to the turbulent bed reactor, and a second catalyst circulation pipeline having an inlet connected to the turbulent bed reactor and an outlet connected to an inlet of the raw material delivery system, wherein:
when the turbulent bed reactor is provided with a first perforated distribution plate and the grids, and the internal circulation line comprises the first catalyst circulation line and the second catalyst circulation line, the outlet of the first catalyst circulation line is connected to the reaction system above the uppermost layer of grid, and the inlet of the second catalyst circulation line is connected to the reaction system between the lowermost layer of grid and the first perforated distribution plate.

12. The fluidized bed reactor according to claim 1, wherein the fluidized bed reactor further comprises a product gas separation system comprising a compression condensing unit, a first separation unit and a second separation unit, wherein:
an inlet of the compression condensing unit is connected to the product gas outlet of the fluidized bed reactor, and a gas phase outlet of the compression condensing unit is connected to an inlet of the first separation unit, and a liquid phase outlet of the compression condensing unit is connected to an inlet of the second separation unit;
the first separation unit is provided with a hydrogen outlet and a light hydrocarbon outlet, and the second separation unit is provided with a propylene outlet, a propane outlet and a fuel hydrocarbon outlet, wherein the light hydrocarbon outlet of the first separation unit is connected to an inlet of the second separation unit; and
the propane outlet of the second separation unit is connected to at least one of the gas-solid separation system, the pre-rising system, and the raw material delivery system in the fluidized bed reactor.

13. A gas-solid airlift loop regenerator, comprising a first regeneration system, a second regeneration system and a first stripper consecutively connected in this order, wherein:
the first regeneration system includes a first casing and a main air distributor, a first ring pipe distributor and a first draft tube arranged inside the first casing, wherein the main air distributor is arranged at the bottom of the first casing, the first draft tube is arranged above the main air distributor, and the first ring pipe distributor is arranged between the first casing and the first draft tube in the horizontal direction, and wherein the top of the first casing is provided with a fuel feed nozzle extending from the outside to the inside of the first casing and located above the first draft tube;
the second regeneration system includes a second casing, a second ring pipe distributor, and a combined cyclone separator located inside the second casing, wherein the second ring pipe distributor is arranged at the bottom of the second casing and below the combined cyclone separator, and wherein the top of the second casing is provided with a gas outlet;
the first stripper is used to remove an oxygen-containing flue gas, and is provided with a gas outlet, an inlet and a solid outlet, wherein the inlet of the first stripper is connected to the bottom of the second casing; and
a regenerant circulation line is connected between the second regeneration system and the first regeneration system.

14. The gas-solid airlift loop regenerator according to claim 13, wherein the vertical distance between the bottom end of the first draft tube and the top end of the first ring pipe distributor is less than or equal to 500 mm, and the vertical distance between the first draft tube and the first main air distributor is greater than or equal to 300 mm, and wherein the vertical distance between the fuel feed nozzle and the first draft tube is 200 mm to 1500 mm.

15. The gas-solid airlift loop regenerator according to claim 13, wherein the second regeneration system further comprises a second draft tube arranged at the inner bottom of the second casing, and wherein the second ring pipe distributor is arranged between the second draft tube and the second casing in the horizontal direction.

16. The gas-solid airlift loop regenerator according to claim 15, wherein the vertical distance between the bottom end of the second draft tube and the second ring pipe distributor is less than or equal to 500 mm.

17. The gas-solid airlift loop regenerator according to claim 13, wherein the gas-solid airlift loop regenerator further comprises a catalyst activator for loading a metal active component onto the regenerant, the catalyst activator being provided with a raw material inlet, a solid outlet, a regenerant inlet and a gas outlet in this order from bottom to top, wherein the regenerant inlet of the catalyst activator being connected to the solid outlet of the first stripper, and wherein a third perforated distribution plate is provided above the raw material inlet of the catalyst activator.

18. A coupled fluidized bed reactor-regenerator apparatus for catalytic dehydrogenation of propane, comprising:
(1) a recirculation inclined pipe;
(2) a regeneration inclined pipe;
(3) the fluidized bed reactor according to claim 1; and
(4) a gas-solid airlift loop regenerator, the gas-solid airlift loop regenerator comprising a first regeneration system, a second regeneration system and a first stripper consecutively connected in this order, wherein:
(a) the first regeneration system includes a first casing and a main air distributor, a first ring pipe distributor and a first draft tube arranged inside the first casing, wherein the main air distributor is arranged at the bottom of the first casing, the first draft tube is arranged above the main air distributor, and the first ring pipe distributor is arranged between the first casing and the first draft tube in the horizontal direction, and wherein the top of the first casing is provided with a fuel feed nozzle extending from the outside to the inside of the first casing and located above the first draft tube;
(b) the second regeneration system includes a second casing, a second ring pipe distributor, and a combined cyclone separator located inside the second casing, wherein the second ring pipe distributor is arranged at the bottom of the second casing and below the combined cyclone separator, and wherein the top of the second casing is provided with a gas outlet;
(c) the first stripper is used to remove an oxygen-containing flue gas, and is provided with a gas outlet, an inlet and a solid outlet, wherein the inlet of the first stripper is connected to the bottom of the second casing; and
(d) a regenerant circulation line is connected between the second regeneration system and the first regeneration system;
wherein:
an inlet of the recirculation inclined pipe is connected to the turbulent bed reactor in the fluidized bed reactor, and an outlet of the recirculation inclined pipe is connected to an inlet of the first regeneration system in the gas-solid airlift loop regenerator; an inlet of the regeneration inclined pipe is connected to the solid outlet of the first stripper, and an outlet of the regeneration inclined pipe is connected to the pre-rising system in the fluidized bed reactor;
when the gas-solid airlift loop regenerator includes the catalyst activator, the solid outlet of the catalyst activator is connected to the inlet of the regeneration inclined pipe, and the gas outlet of the catalyst activator is connected to the gas-solid separation system in the fluidized bed reactor;
when the fluidized bed reactor includes the product gas separation system, the fuel hydrocarbon outlet of the second separation unit in the product gas separation system is connected to the fuel feed nozzle of the first regeneration system; and
when the gas-solid airlift loop regenerator includes the catalyst activator and the fluidized bed reactor includes the product gas separation system, the raw material inlet of the catalyst activator is connected to the propane outlet of the second separation unit.

19. The coupled fluidized bed reactor-regenerator apparatus for catalytic dehydrogenation of propane according to claim 18, wherein when the gas-solid airlift loop regenerator includes the catalyst activator and the fluidized bed reactor includes the product gas separation system, the product gas separation system further includes a primary condensation unit and a gas-solid separation unit, and the product gas outlet of the fluidized bed reactor, the primary condensation unit, the gas-solid separation unit and the compression condensing unit are consecutively connected in this order.

20. The coupled fluidized bed reactor-regenerator apparatus for catalytic dehydrogenation of propane according to claim 18, wherein:
a part of the catalyst in the turbulent bed reactor is delivered into the recirculation inclined pipe, and the remaining catalyst entrained by the gas is delivered from the turbulent bed reactor to the gas-solid separation system for gas-solid separation;
separated gas is discharged from the gas outlet of the gas-solid separation system, allowing the separated catalyst to fall to the bottom of the casing of the gas-solid separation system to form a dense phase bed, and
the internal circulation pipeline is used to deliver the catalyst in the dense phase bed to the raw material delivery system and/or the reaction system for recycling.
